(19) United States
(12) Patent Application Publication
(10) Pub. No.: US 2008/0081045 A1
(43) Pub. Date: Apr. 3, 2008

(54) Combination Therapy for Cancer

(75) Inventors: Mairead Kehoe-Whistance, Ringoes, NJ (US); Grant MacLean, Victoria (CA)

Correspondence Address: BROWDY AND NEIMARK, P.L.L.C.;624 NINTH STREET, NW, SUITE 300, WASHINGTON, DC, 20001-5303, US

(73) Assignee: BIOMIRA, INC., Edmonton (CA)

(21) Appl. No.: 10/575,618

(22) PCT Filed: Oct. 14, 2004
(86) PCT No.: PCT/US04/33988

§ 371 Date: Jul. 17, 2007

Related U.S. Application Data
(60) Provisional application No. 60/510,516, filed on Oct. 14, 2003.
(60) Provisional application No. 60/576,624, filed on Jun. 4, 2004.

(51) Int. Cl. A61K 39/00; A61K 31/135; A61P 35/00; A61K 31/335

(52) U.S. Cl. 424/193.1; 514/449; 514/649

(57) ABSTRACT

Anti-hormonal (anti-estrogenic steroid) therapy and immunotherapy are used in combination to treat breast cancer. The preferred immunotherapeutic agent is an immunogen, preferably comprising sialyl-Tn, and more preferably is a sialyl-Tn/KLH conjugate.

This application claims the benefit under 35 USC 119(e) and under the Paris Convention of Kehoe-Whistance and Maclean, U.S. Ser. No. 60/510,516 filed Oct. 14, 2003, atty docket MACLEAN=1 (originally filed in the name of Maclean alone); Kehoe-Whistance, U.S. Ser. No. 60/576,624 filed Jun. 4, 2004, atty docket KEHOE=1. Both applications are hereby incorporated by reference in their entirety.

### BACKGROUND OF THE INVENTION

1. Field of the Invention

The field of the present invention relates to an anti breast cancer therapy. The present invention contemplates methods and compositions for the combination of immunotherapy and anti-hormone (anti-estrogenic steroid) therapy for this purpose.

2. Description of the Background Art

### Breast Cancer

Breast cancer is the most frequently diagnosed cancer of women in Canada. Over the course of a lifetime, one in nine women is expected to develop breast cancer and one in twenty seven women will die from it. Despite advances in the diagnosis and treatment of breast cancer, up to 50% of newly diagnosed patients may develop metastases. Metastatic breast cancer has a poor prognosis and is generally considered incurable. Typically, the goals of therapy in the setting of metastatic breast cancer are to control the disease and relieve symptoms as much as is possible to maintain or improve quality of life. It has been estimated that the “residual life expectancy” (the additional time a person with cancer would have lived in the absence of the disease) of a woman with breast cancer is 19.6 years. There is a clear and unmet clinical need to develop effective therapeutics for the prolongation of life and relief symptoms for women with metastatic breast cancer.

### Risk Factors in Breast Cancer

There a variety of risk factors that are important in the etiology of breast cancer. These include: increasing age (risk doubles between the ages of 45 and 65), previous breast cancer in the same patient, family history of breast cancer in a first degree relative (mother, sister or daughter), a first degree relative that is pre-menopausal and has bilateral breast cancer, a first degree relative that is pre-menopausal and has uni-lateral breast cancer, and a blood relative that is pre-menopausal and has breast cancer. Additional risk factors include: carriers of mutations of genes such as BRCA1, BRCA2, p53, PTEN, ATM, a family history of cancer of the ovary, cervix, uterus or colon, early menarche, late menopause, nulliparity, first pregnancy over the age of 30, obesity, breast augmentation, oral contraceptives, hormone replacement therapy (HRT), and radiation exposure. Diet and alcohol consumption may also affect the risk of developing breast cancer and this may, in part, explain the fivefold variation in the incidence of breast cancer that is observed among different countries.

### Screening and Diagnosis of Breast Cancer

Screening for the presence of breast cancer can be carried out in a variety of ways, including: beast self-examination, clinical breast examination, mammography, and screening mammography. Evaluation of abnormalities detected during screening can be carried out by: fine-needle aspiration, ultrasonography, biopsy, mammography, stereotactic- and ultrasound-guided core biopsies, ultrasound- or stereotactic-guided fine-needle aspiration, magnetic resonance imaging, ultrasound, sestamibi nuclear medicine scanning and positron emission tomography imaging. The invention is not limited to a particular method of screening for the presence of breast cancer.

### Markers of Breast Cancer

Her-2/neu and c-erB-2 are receptor protein tyrosine kinases that members of the epidermal growth factor receptor (EGFR) family. Over expression of growth factor receptors with homology to EGFR (such as Her-2/neu and/or c-erB-2) have been found to be associated with a poor clinical prognosis of beast carcinoma. It has been noted that those breast carcinomas overexpressing Her2/neu and/or c-erB-2 tend to lack the estrogen and progesterone receptors, and thus are hormone-therapy less-responsive (see below), and have a poor clinical outcome. The invention does not require screening for these markers of breast cancer.

### Staging of Breast Cancer

A widely used system to stage breast cancer is the American Joint Committee on Cancer (AJCC) classification, which is based on tumor size (T), the status of regional lymph nodes (N), and the presence of distant metastasis (M), and is referred to as TNM staging. Clinical staging is performed following physical examination and radiological studies. Pathologic staging is performed following surgery for operable breast cancer The stage of the cancer may influence the choice of treatment a skilled clinician may offer to a patient.

### Treatment of Metastatic Breast Cancer

Due to the heterogeneity of metastatic breast cancer there are a variety of treatment options for these patients which include, but are not limited to: surgery, chemotherapy, radiation therapy, hormonal therapy and immunotherapy. The preferred treatment regime will depend on factors such as extent of metastases, comorbid condition and tumour characteristics. Such factors that contribute to the treatment regime are well known to the skilled practitioner. Treatment guidelines relating to various drug products are also well known to the skilled practitioner, and, by way of example, may be found in the “Compendium of Pharmaceuticals and Specialties (CPS), The Canadian Drug Reference for Health Professionals”, and other such guides.

### Immunotherapy of Breast Cancer

WO 03/015796 (“Immunogenic conjugate of carbohydrate haptens and aggregated protein carrier”) describes an immunotherapy in which an immune response is elicited to a carbohydrate epitope. In particular, an aggregated STn-KLH (keyhole limpet hemocyanin) conjugate, also known as the THERATOPE® vaccine, is described.

The THERATOPE® vaccine developed at Biomira consists of a synthetic STn hapten conjugate to KLH, delivered in emulsion with an adjuvant. The vaccine used in Phase I and Phase II clinical trials had a hapten substitution level that resulted in a sialic acid (NANA) content of about 2.5 to 3% by weight. While Phase II clinical trials were in progress, the conjugation methodology was improved so that a NANA content of about 7$ could be achieved. The high conjugation product induced considerably higher titers of anti-Stn antibody in mice, and significantly higher anti-STn IgG titers in humans, in a small bridging study. Since higher anti-STn IgG titers had appeared to be correlated with improved survival in Phase II clinical trials, a large phase III clinical trial was initiated using a STn-KLH conjugate with a NANA content of about 7%. Further refinements of the STn-KLH conjugate are described in PCT/US02/24735, filed Aug. 5, 2002 (atty docket KRANTZ2.1-PCT).

### SUMMARY OF THE INVENTION

The present invention contemplates the use of anti-hormonal (anti-estrogenic steroid) therapy in combination with immunotherapy as an anti-breast cancer therapy, and kits for practicing said combination therapy. It further relates to methods of identifying patients likely to benefit from such therapy.

The immunotherapy comprises administration of at least one immunotherapeutic agent, which may comprise an immunogen (active immunotherapy), an antibody (passive immunotherapy), or an antigen-activated T cell (passive immunotherapy). The immunogen comprises at least one breast-cancer associated epitope, and the antibody or T cell recognizes at least one breast cancer-associated epitope.

In a preferred embodiment, the breast cancer-associated epitope is a carbohydrate epitope, in particular, the TF, sialyl-TF, Tn or, most preferably, the sialyl-Tn epitope. Even more preferably, the immunogen is an Stn-KLH conjugate. Most preferably, the immunotherapeutic agent is the THERATOPE® vaccine.

The breast cancer-associated epitope may alternatively be a peptide or glycopeptide epitope, in which case it preferably is a MUC1 epitope.

It should be noted that MUC1 comprises Sialyl Tn epitopes as well as peptide epitopes.

By anti-hormonal (anti-estrogenic steroid) therapy is meant therapy which comprises administration of an agent which inhibits an endogenous hormone (a human estrogenic steroid) which, at normal or elevated levels of activity, is a risk factor for breast cancer.

### BRIEF DESCRIPTION OF THE DRAWINGS

FIG. 1 Survival of THERATOPE® vaccine versus KLH Control groups for Fully Adjusted Hormone Subset: original Data. Survival distribution function plotted against survival time (months).

FIG. 2 Median Survival for Fully Adjusted Hormone Subset as of Update #2. Survival distribution function plotted against survival time (months).

FIG. 3 Survival by OSM Response for the Fully Adjusted Hormone Subset of THERATOPE® vaccine Treated Patients, as of Update #2. Survival distribution function plotted against survival time (months).

### DETAILED DESCRIPTION OF THE PREFERRED EMBODIMENTS OF THE INVENTION

The present invention relates to the use of anti-hormonal (anti-estrogenic steroid) therapy in combination with immunotherapy (such as with the THERATOPE® vaccine) as an anti-breast cancer therapy.

### Estrogenic Steroids

The contemplated endogenous human estrogenic steroids are principally the human estrogens 17beta-estradiol, estrone and estriol. We use the term “estrogenic steroid” instead of “estrogen” principally because some scientists use the term “anti-estrogen” to refer to compounds which antagonize estrogen by a particular mechanism. The term “anti-estrogenic steroid” is not limited to any particular mechanism of action.

### Hormones and Breast Cancer

Hormones, such as estrogen, play an important role in the progression of breast cancer. In premenopausal women, estrogen (17beta-estradiol) is produced predominantly (although not exclusively) in the ovaries through aromatization of estrogen precursors (such as androstenedione) catalyzed by the enzyme estrogen synthetase (aromatase). In menopausal women the ovaries no longer produce estrogen. However, aromatization of adrenal androgen can still occur in peripheral tissues, resulting in the production of 17beta-estradiol.

Estrogens are involved in cellular proliferation and the maintenance of breast tissue. The proliferative effects of estrogens are also involved in the promotion of tumor growth in breast cancer, and a number of therapeutic approaches designed to reduce the amounts of estrogen have been developed.

In premenopausal women, removal of the ovaries (by oophorectomy, radiation therapy, or biochemical castration) reduces the amount of estrogen and thereby reduces the proliferative effects of estrogen on tumour growth. In women without functional ovaries, antiestrogens and aromatase inhibitors can be used to reduce the amount of estrogen that is produced in the peripheral tissues.

Anti-Hormonal (Anti-Estrogenic Steroid) Therapy

An antihormonal therapeutic agent is a chemical agent which reduces the level of estrogen in the body, or which antagonizes estrogen activity. The term “chemical agent” is used to exclude radiation therapy or surgery. Of course, these treatments may be used in addition to the contemplated chemical treatment. The chemical agent may be a biochemical, such as an enzyme or hormone. (Note that some hormones inhibit the actions of other hormones.)

A chemical agent can reduce the level of estrogen in an organ or tissue by inhibiting the synthesis or secretion by estrogen-producing cells, or the transport of estrogen to the organ or tissue of interest. Or it can promote the catabolism of estrogen. A chemical agent can also be used to reduce estrogen activity, e.g., by inhibiting the binding of estrogen to a receptor (pharmacological antagonism), or by inhibiting a downstream activity resulting from activation of an estrogen receptor (physiological antagonism).

Treatment with antiestrogens and/or aromatase inhibitors can be referred to either as “hormonal therapy” or as “antihormonal therapy”, although the latter term is preferred. Reference to anti-hormonal therapy, unless otherwise specified, should be taken as referring to anti-estrogenic steroid therapy.

Hormonal-therapy and antihormonal-therapy should not be confused with “hormone replacement therapy” (HRT) which is a controversial medical treatment for women with symptoms (hot flashes, night sweats etc.) associated with menopause.

For the purpose of the present claims, the term “anti-hormonal therapy” includes (but is not limited to) treatment with antiestrogens, aromatase inhibitors and/or functionally equivalent compounds as, by way of example, described herein below.

Antiestrogens. Antiestrogens are used as a therapy in the treatment of metastatic breast cancer and inhibit estrogen-induced proliferation through interaction with the estrogen receptor. One mechanism is by competitive inhibition of the estrogen receptor, and hence the “antiestrogens” include estrogen receptor antagonists and SERMs (see below). It has also been suggested that antiestrogen/estrogen-receptor complex can inhibit transcription of genes that are under control of the estrogen-response elements. As a result of this reduced gene transcription, cell division is also reduced.

Examples of non-steroidal antiestrogens include toremifene, tamoxifen, droloxifene and trioxifene. Fulvestrant is an example of a steroidal antiestrogen.

Estrogen receptor antagonists. The anti-estrogenic steroid compound may be an estrogen receptor antagonist. That is, it competitively inhibits the binding of estrogen to at least one estrogen receptor, without significantly activating the receptor in its own right. ICI182,780 is the best known pure antagonist.

A large number of peptides which bind and selectively inhibit an estrogen receptor have been identified by combinatorial library techniques, i.e., by screening a combinatorial library for those which bind a particular estrogen receptor target. See Norris J D, Paige L A, Christensen D J, et al.: Peptide antagonists of the human estrogen receptor. Science 1999, 285:744-746. Based on knowledge of these peptides, peptidomimetics can be designed.

Selective Estrogen Receptor Modulators (SERMs). SERMs are compounds which competitively inhibit the binding of estrogen to one or more of the estrogen receptors. There are several different estrogen receptors, and the SERMs vary with respect to their spectrum of affinity for the receptors.

Unlike a pure estrogen receptor antagonist, the SERMs activate one or more of the estrogen receptors to which they bind. In other words, they are agonists with respect to at least one estrogen receptor, and antagonists with respect to at least one other estrogen receptor. Consequently, they have some estrogen-like activity, but their spectrum of activation is different from that of the human estrogens. In other words, SERMs are “designer” estrogens. Clearly, the SERMs of interest are those whose estrogen-like activities do not include the activities which are cancer growth-promoting.

Since the estrogen receptors vary from tissue to tissue, the effects of SERMs may also vary from one tissue to the next. We are concerned with the effect of SERMs on breast cancer tissue, as well as with any side effects, desirable or undesirable, in any tissue. Preferably, the SERM prevents bone loss (like estrogen) and lowers serum cholesterol (like estrogen). SERMs of interest include tamoxifen (most preferred), toremifene, droloxifen, clomifene, arzoxifene, raloxifene, the raloxifene analog LY 117018 and SERM EM-652.

It should be noted that a compound, especially a steroid, which initially appears to be a pure estrogen receptor antagonist may prove on closer examination to act as an agonist for some estrogen receptors, in which case it is reclassified as a SERM. Compounds noted here as being as interest as estrogen receptor antagonists remain of interest even if they are so reclassified.

Just as combinatorial libraries can be used to identify peptides which act as pure estrogen receptor antagonists, they can be used to identify SERMS. All that is necessary is that peptides which bind one estrogen receptor be screened against additional estrogen receptors, and the peptides which bind a plurality of receptors are then screened for agonist/antagonist activity. One successful peptide SERMs are identified, peptidomimetic SERMs can be designed.

Estrogen receptor disruptors. Drugs may inhibit the synthesis of, permanently inactivate, or even destroy estrogen receptors. Fulvestrant has this activity.

Aromatase inhibitors. Aromatase inhibitors reduce the levels of estrogens by inhibiting the aromatase enzyme complex, which is responsible for synthesizing estrogen. Examples of aromatase inhibitors include aminoglutethimide*, anastrozole**, vorozole, letrozole**, liarozole, megastrole, exemestane*, and formestane* (* is preferred, ** is most preferred). Naturally, combinatorial libraries can be used to identify peptides which bind to aromatase, and these peptides further screened for inhibitory activity.

While in the discussions above, we have referred to combinatorial peptide libraries, it should be noted that these teachings apply, mutatis mutandis, to the screening of other combinatorial libraries for active compounds, using estrogen receptors or aromatase as the binding target.

Miscellaneous Anti-Estrogenic Steroid Agents. There are, of course, other compounds whose mechanisms of action as less well elucidated, yet can be considered anti-hormonal therapy. Such compounds can include geoselin acetate (Zoladex®) and megestrol acetate (Megase®).

### The Immune System.

The ability of vertebrates to protect themselves against infectious microbes, toxins, viruses, or other foreign macromolecules is referred to as immunity. Acquired or specific immunity comprises defense mechanisms which are induced or stimulated by exposure to foreign substances.

The events by which the mechanisms of specific immunity become engaged in the defense against invading microorganisms cancer cells, etc. are termed immune responses. Vertebrates have two basic immune responses: humoral and cellular. Humoral immunity is provided by B lymphocytes, which, after proliferation and differentiation, produce antibodies which circulate in the blood and lymphatic fluid. Cellular immunity is provided by the T cells of the lymphatic system. The cellular immune response is particularly effective against fungi, parasites, intracellular viral infections, cancer cells and foreign matter, whereas the humoral response primarily defends against the extracellular phases of bacterial and viral infections.

An “antigen” is a foreign substance which is recognized (specifically bound) by an antibody or a T-cell receptor, regardless of whether it can induce an immune response. Foreign substances inducing specific immunity are termed “immunizing antigens”, or “immunogens”. An “hapten” is an antigen which cannot, by itself, elicit an immune response (though a conjugate of several molecules of the hapten, or of the hapten to a macromolecular carrier, might do so). Since the present application is concerned with eliciting immune response, the term “antigen” will refer to immunizing antigens unless otherwise stated.

### Immunological Agents

An immunological agent is one which contributes to acquired immunity, actively or passively, and hence comprises at least one of the following: an immunogen, an antibody, or an antigen-activated T cell.

### Anti-Breast Cancer Immunogen

The immunogen of the present invention is a molecule comprising at least one breast cancer-associated B or T cell epitope, as defined below, and which, when suitably administered to a subject (which, in some cases, may mean associated with a liposome or with an antigen-presenting cell), elicits a humoral and/or cellular immune response which is protective, at least as part of the contemplated combination therapy, against breast cancer.

### Breast Cancer-Associated Epitope

The epitopes of the present invention may be directly or indirectly associated with breast cancer, with the former being preferred.

An epitope may be said to be directly associated with breast cancer if it is presented by an intracellular, surface or secreted antigen of breast cancer. It need not be presented by all breast cancer cell lines, or by all cells of a particular tumor, or throughout the entire life of the tumor. It need not be specific to the tumor in question.

An epitope may be indirectly associated with breast cancer if the epitope is of an antigen which is specifically produced or overproduced by non-breast tumor cells of the subject in specific, but non-immunological, response to the disease, e.g., an angiogenic factor which is overexpressed by nearby cells as a result of regulatory substances secreted by a tumor.

The term “breast cancer associated epitope” also includes any non-naturally occurring epitope which is sufficiently similar to an epitope naturally associated with the breast cancer so that antibodies or T cells which recognize the natural epitope also recognize the similar non-natural epitope.

Preferably, the epitope is not merely associated with breast cancer, but is specific to breast cancer. An epitope may be said to be specific to breast cancer, if it is associated more frequently with that source than with other sources, to a detectable and clinically useful extent. Absolute specificity is not required, provided that a useful prophylactic, therapeutic or diagnostic effect is still obtained.

A breast cancer specific epitope is more frequently associated with that tumor that with other tumors, or with normal cells. Preferably, there should be a statistically significant (p=0.05) difference between its frequency of occurrence in association with breast cancer, and its frequency of occurrence in association with (a) normal breast cells, and (b) at least one other type of tumor.

The term “breast cancer specific epitope” also includes any non-naturally occurring epitope which is sufficiently similar to a naturally occurring epitope specific to breast cancer so that antibodies or T cells stimulated by the similar epitope will be essentially as specific as CTLs stimulated by the natural epitope.

In general, tumor-versus-normal specificity is more important than tumor-versus-tumor specificity as (depending on the route of administration and the particular normal tissue affected), higher specificity generally leads to fewer adverse effects. Tumor-versus-tumor specificity is more important in diagnostic as opposed to therapeutic uses.

The term “breast cancer specific” is not intended to connote absolute specificity, merely a clinically useful difference in probability of occurrence in association with breast cancer rather than in a matched normal subject.

The epitopes of the present invention may be B-cell or T-cell epitopes, and they may be of any chemical nature, including without limitation, peptides, carbohydrates, lipids, glycopeptides and glycolipids. The epitope is at least substantially the same as a naturally occurring epitope. It may be identical to a naturally occurring epitope, or a modified form of a naturally occurring epitope.

A term such as “breast cancer-associated epitope” includes both native and mutant epitopes, but the mutant epitope must be cross-reactive with a native breast cancer-associated epitope. Likewise, “MUC1 epitope”, without further qualification, is intended to encompass, not only a native epitope of MUC1, but also a mutant epitope which is substantially identical to a native epitope. Such a mutant epitope must be cross-reactive with a native MUC1 epitope.

For methods of identifying naturally occurring epitopes in a natural antigen, or of predicting the B and T cell epitopes in a polypeptide antigen for which the amino acid sequence is known, or of designing a mutant epitope based on a known natural epitope, see PCT/US03/10750, filed Apr. 9, 2003 (atty docket KOGANTY4A-PCT).

### B-Cell Epitopes

B-cell epitopes are epitopes recognized by B-cells and by antibodies.

B-cell peptide epitopes are typically at least five amino acids, more often at least six amino acids, still more often at least seven or eight amino acids in length, and may be continuous (“linear”) or discontinuous (“conformational”) (the latter being formed by the folding of a protein to bring noncontiguous parts of the primary amino acid sequence into physical proximity).

B-cell epitopes may also be carbohydrate epitopes.

### T cell Epitopes

A T cell epitope is one which can stimulate or enhance a cellular immune response to that epitope. The epitope must, of course, be one amenable to recognition by T-cell receptors so that a cellular immune response can occur. For peptides, the T-cell epitopes may interact with class I or class II MHC molecules. The class I epitopes usually 8 to 15, more often 9-11 amino acids in length. The class II epitopes are usually 5-24 (a 24 mer is the longest peptide which can fit in the Class II groove), more often 8-24 amino acids. If the immunogen is larger than these sizes, it will be processed by the immune system into fragments of a size more suitable for interaction with MHC class I or II molecules.

The carbohydrate T-cell epitopes may be as small as a single sugar unit (e.g., Tn). They are preferably no larger than five sugars.

Many T-cell epitopes are known. Several techniques of identifying additional T-cell epitopes are recognized by the art. In general, these involve preparing a molecule which potentially provides a T-cell epitope and characterizing the immune response to that molecule. Methods of characterizing the immune response are discussed in Ser. No. PCT/US03/10750, filed Apr. 9, 2003, atty docket KOGANTY4A-PCT.

The reference to a CTL epitope as being “restricted” by a particular allele of MHC Class I molecules, such as HLA-A1, indicates that such epitope is bound and presented by the allelic form in question. It does not mean that said epitope might not also be bound and presented by a different allelic form of MHC, such as HLA-A2, HLA-A3, HLA-B7, or HLA-B44.

### Carbohydrate Epitopes

The epitope of the present invention may be a carbohydrate epitope. The Tn, T, sialyl Tn (STn) and sialyl (2->6)T epitopes are particularly preferred. The STn epitope is the most preferred.

The term “carbohydrate” includes monosaccharides, oligosaccharides and polysaccharides, as well as substances derived from the monosaccharides by reduction of the caronyl group (alditols), by oxidation of one or more terminal groups to carboxylicacids, or by replacement of one or more hydroxy groups by a hydrogen atom, an amino group, a thiol group, or similar heteroatomic groups. It also include derivatives of the foregoing.

The epitope may be part of a carbohydrate hapten. Normally, a carbohydrate hapten will not be a polysaccharide, as a polysaccharide is usually large enough to be immunogenic in its own right. The borderline between an oligosaccharide and a polysaccharide is not fixed, however, we will define an oligosaccharide as consisting of 2 to 20 monosaccharide (sugar) units.

The hapten may be a monosaccharide (without glyosidic connection to another such unit) or an oligosaccharide. If an oligosaccharide, it preferably is not more than 10 sugar units.

Monosaccharides are polyhydroxy aldehydes (H[CHOH]ₙ—CHO) or polyhydroxy ketones (H—[CHOH]ₙ—CO—[CHOH]ₘ—H) with three or more carbon atoms.

Each monosaccharide unit may be an aldose (having an aldehydic carbonyl or potential aldehydic carbonyl group) or a ketose (having a ketonic carbonyl or potential ketonic carbonyl group). The monosaccharide unit further may have more than one carbonyl (or potential carbonyl) group, and hence may be a dialdose, diketose, or aldoketose. The term “potential aldehydic carbonyl group” refers to the hemiacetal group arising from ring closure, and the ketonic counterpart (the hemiketal structure).

The monosaccharide unit may be a cyclic hemiacetal or hemiketal. Cyclic forms with a three membered ring are oxiroses; with four, oxetoses, with five, furanoses; with six, pyranoses; with seven, septanoses, with eight, octaviruses, and so forth. The locants of the positions of ring closure may vary.

The monosaccharide unit may further be a deoxy sugar (alcoholic hydroxy group replaced by hydrogen), amino sugar (alcoholic hydroxy group replaced by amino group), a thiosugar (alcoholic hydroxy group replaced by thiol, or C═) replaced by C═S, or a ring oxygen of cyclic form replaced by sulfur), a seleno sugar, a telluro sugar, a (-substituted monosaccharide, an unsaturated monosaccharide, an aza sugar (ring carbon replaced by nitrogen), an amino sugar (ring oxygen replaced by nitrogen) an alditole (carbonyl group replaced with CHOH group), aldonic acid (aldehydic group replaced by carboxy group), a ketoaldonic acid, a uronic acid, an aldaric acid, and so forth.

Sialic acid, also known as N-acetyl neuraminic acid (NANA), is of particular interest. It is the terminal sugar on several tumor-associated carbohydrate epitopes.

### Mucin Epitope

Numerous antigens of clinical significance bear carbohydrate determinants. One group of such antigens comprises the tumor-associated mucins (Roussel, et al., Biochimie 70, 1471, 1988).

In a preferred embodiment, the epitope is an epitope of a cancer-associated mucin. Generally, mucins are glycoproteins found in saliva, gastric juices, etc., that form viscous solutions and act as lubricants or protectants on external and, internal surfaces of the body. Mucins are typically of high molecular weight (often >1,000,000 Dalton) and extensively glycosylated (over 80%). The glycan chains of mucins are O-linked (to serine or threonine residues) and may amount to more than 80% of the molecular mass of the glycoprotein.

Mucins are produced by ductal epithelial cells and by tumors of the same origin, and may be secreted, or cell-bound as integral membrane proteins (Burchell, et al., Cancer Res., 47, 5476, 1987; Jerome, et al., Cancer Res., 51, 2908f 1991).

Cell membrane mucins have distinct external, transmembrane, and cytoplasmic domains. They exist as flexible rods and protrude relatively great distances from the cell surface forming an important component of the glycocalyx (Jentoff, 1990) and the terminal carbohydrate portions thereof are probably the first point of contact with antibodies and cells of the immune system.

Cancerous tissues produce aberrant mucins which are known to be relatively less glycosylated than their normal counterparts (Hull, et al., Cancer Commun., 1, 261, 1989). Due to functional alterations of the protein glycosylation machinery in cancer cells, tumor-associated mucins typically contain short, incomplete glycans.

Thus, while the normal mucin associated with human milk fat globules consists primarily of the tetrasaccharide glycan, gal beta1-4 glcNAcpl-(gal beta1-3) gal NAc-alpha and its sialylated analogs (Hull, et al.), the tumor-associated Tn hapten consists only of the monosaccharide residue, alpha-2-acetamid-2-deoxy-D-galactopyranosyl, and the T-hapten of the disaccharide beta-D-galactopyranosyl-(1-3)alpha-acetamido-2-deoxy-D-galactopyranosyl.

Other haptens of tumor-associated mucins, such as the sialyl-Tn and the sialyl-(2-6)T haptens, arise from the attachment of terminal sialyl residues to the short Tn and T glycans (Hanisch, et al., Biol. Chem. Hoppe-Seyler. 370, 21, 1989; Hakormori, Adv. Cancer Res., 52:257, 1989; Torben, et al., Int. J. Cancer, 45 666, 1980; Samuel, et al., Cancer Res., 50, 4801-1990).

Because of these differences in glycosylation, the aberrant cancer-associated mucins are antigenically different from their normal cell counterpart mucins, exposing normally cryptic carbohydrate—(Hanish et al, 1989; Torben et al, 1990; Samuel et al, 1990), peptide-(Burchell et al, 1987) and perhaps even glycopeptide-epitopes. Therefore, because cell surface mucins protrude, they themselves may serve as targets for immune attack (Henningson, et al., 1987; Fung, et al., 1990; Singhal, et al., 1991; Jerome et al., 1991; Oncogen, EP 268,279; Biomembrane Institute, WO89/08711; Longenecker, U.S. Pat. No. 4,971,795). Under some circumstances, cancer-associated cell membrane mucins can actually “mask” other cell surface antigens and protect cancer cells from immune attack (Codington et al, 1983; Friberg, 1972; Miller et al, 1977).

The T and Tn antigens (Springer, Science, 224, 1198, 1984) are found in immunoreactive form on the external surface membranes of most primary carcinoma cells and their metastases (>90% of all human carcinomas). As cancer markers, T and Tn permit early immunohistochemical detection and prognostication of the invasiveness of some carcinomas (Springer). The presence of the sialyl-Tn hapten on tumor tissue has been identified as an unfavorable prognostic parameter (Itzkowitz, et al. Cancer, 66, 1960, 1990; Yonezawa, et al., Am. J. Clin. Pathol., 98 167, 1992).

The altered glycan determinants displayed by the cancer associated mucins are recognized as non-self or foreign by the patient's immune system (Springer). Indeed, in most patients, a strong autoimmune response to the T hapten is observed. These responses can readily be measured, and they permit the detection of carcinomas with greater sensitivity and specificity, earlier than has previously been possible. Finally, the extent of expression of T and Tn often correlates with the degree of differentiation of carcinomas (Springer).

The mucin epitope may be a core peptide, a carbohydrate, or a glycopeptide (Consequently, there is overlap between the mucin epitope and carbohydrate epitope embodiments of the invention.) Non-limiting examples of mucins which may carry epitopes are the human tumor associated Thomsen-Friedenreich antigen, (MacLean, 1992), epiglycanin-related glycoprotein (Codington, 1984) ovine submaillary mucin, bovine submaxillary mucin, breast tumor mucins (e.g., human polymorphic epithelial mucin, including breast tumor mucins, Gendler, 1988, 1990; breast cancer epithelial tumor antigen, Hareuveni, 1990, breast carcinoma, Hull, 1989), mammary tumor mucins (e.g., such as murine mammary adenocarcinoma, Fung, 1990) carcinoma mucins such as mucins arising from the kidney (e.g., renal cell carcinoma), ovary (e.g., ovarian carcinoma-associated sebaceous gland antigen, Layton, 1990), bladder, colon (e.g., Sialosyl-Tn in colorectal cancer, Itzkowitz, 1990) pancreatic tumor mucin (Lan, 1990), gallbladder, bladder, colon (e.g., malignant colon mucosa mucins, Torbin, 1980) and some lung tissues, melanoma mucins (e.g., melanoma-associated antigen, Kahn, 1991) epithelial tumor cell mucins, leukemia associated mucins, carcinoembryonic antigen, or any other mucin associated with abnormal cells according to known characteristics of cancer associated mucins or abnormal mucins, such as aberrant glycosylation (Hakomori, 1989, and Singhal, 1990).

### MUC1 Epitopes

The human MUC1 gene product has been referred to by various names, including MAM6, milk mucin; human milk fat globule antigen (HMFG); human mammary epithelial antigen, CA 15-3, CA 27.29; episialin; and polymorphic epithelial mucin (PEM) (reviewed in Taylor-Papadimitriou et al, 1988)(for complete cites to the incompletely cited references in this section, see Longenecker, et al., 08/229,606). This mucin is strongly expressed on human breast (Gendler et al, 1988), pancreatic (Lan et al, 1990) and certain ovarian cancer cells (Layton et al, 1990). Although the MUC1 encoded mucins expressed on various cancers contain the same tandem repeat core peptide sequence, glycosylation differences do exist (Gendler et al, 1988; Lan et al, 1990). Because of under-glycosylation in cancer cells, MUC-1 molecules on cancer cells express cryptic epitopes which are not expressed (i.e, are cryptic) on normal epithelial cells.

MUC1 is the first cancer-associated mucin gene to be cloned and mapped (Gendler et al, 1990), and has recently been transfected into a murine mammary cell line, 410.4 (Lalani et al, 1991). MUC1 transfected 410.4 cells express the MUC1 gene product on the cell surface.

The pattern of glycosylation is similar to, but different from, malignant cell derived mucins expressing the same cryptic peptide epitopes as expressed by human cancer associated MUC1 (Taylor-Papadimitriou et al, 1988). Lalani and co-workers (1991) have examined the immunogenicity of the 410.4 transfectants in mice. These workers demonstrated that mice which rejected a low dose of transfected 410.4 cells did not develop tumors after a subsequent transplant of a high dose of transfected 410.4 cells although no effect on tumor development of untransfected wild type 410.4 cells was seen (Taylor-Papadimitriou et al, 1988). (For complete cites, see PCT/US95/04540, filed Apr. 12, 1995 (atty docket LONGENECKER5-PCT), and see also refs 4-11 thereof).

It has been shown that cancer vaccines composed of synthetic peptide antigens which mimic cryptic MUC-1 peptide sequences on cancer cells are able to induce effective anti-cancer immunotherapy against MUC-1 expressing tumor cells in a murine model Finn and co-workers have shown that cancer patients are able to produce specific non-MHC restricted cytotoxic T-lymphocytes (CTL) which recognize peptide epitopes expressed on MUC-1 molecules on cancer cells. (See refs. 12 and 53-55 of Longenecker5-PCT). Indeed the MUC1 sequence SAPDTRP (AAs 4-10 of SEQ ID NO:1) has been shown to be both a T- and a B-cell epitope. It has been demonstrated that the immunization of chimpanzees with synthetic MUC-1 antigens induces the development of specific antibodies and CMI against MUC-1.

The human epithelial mucin MUC1 is over-expressed in more than 90% of carcinomas of the breast, ovary and pancreas, and in those tumors it is aberrantly glycosylated. The SM3 antibody binds the core protein of MUC1; it also binds the tumor glycoproteins, presumably because the SM3 epitope is exposed as a result of the aforementioned aberrant glycosylation.

The amino acid sequence of Human MUC1 is available in the SWISS-PROT database as P15941. The number of repeats is highly polymorphic. It varies from 21 to 125 in the northern european population. The most frequent alleles contains 41 and 85 repeats. The tandemly repeated icosapeptide underlies polymorphism at three positions, as shown by brackets: PAPGSTAP[P/A/Q/T]AHGVTSAP[D/E] [T/S]R (SEQ ID NO:2). The common polymorphisms are the coordinated double mutation DT ->ES and the single replacements P ->A, P ->Q and P->T. The most frequent replacement DT>ES occurs in up to 50% of the repeats. For Mouse MUC1, see SWISS-PROT Q02496.

Moller, et al., Eur. J. Biochem. 269:1444-55 (March 2002) has used NMR spectroscopy to study the binding of the SM3 antibody to the pentapeptide MUC1 epitope PDTRP and to the related glycopentapeptide in which the threonine is O-lined to alpha-d-GalNAc. Moller found that the PDT interacted with the SM3 antibody more strongly than did the RP, suggesting that the RP would be more tolerant of mutation. In contrast, the glycopeptide interacted with SM3 using all of its amino acids, although the strongest effect was with the Pro1. Docking studies were conducted; these could be performed with mutant peptides for which 3D structures are deducible or determined.

Hiltbold, et al., Cancer Res., 58:5066-70 (1998) showed that CD4+ T-cells primed in vitro with a synthetic MUC1 peptide of 100 amino acids, representing five unglycosylated tandem repeats, and presented by dendritic cells, produced IFN-gamma and had moderate cytolytic activity. They also identified a core peptide sequence, PGSTAPPAHGVT (SEQ ID NO:3), which elicits this response when it is presented by HLA-DR3.

Heukamp, et al., Int. J. Cancer, 91:385-92 (2001) eleicted peptide-specific CTL immunity in A2/K(b) transgenic mice with three MUC1-derived peptides that map outside the variable number tandem repeat region. These peptides were MUC(79-87) (TLAPATEPA) (SEQ ID NO:4), MUC(167-175) (ALGSTAPPV) (SEQ ID NO:5) and MUC(264-72) (FLSFHISNL) (SEQ ID NO:6). All comply with the peptide binding motif for HLA-A*0201.

Engelmann, et al., J. Biol. Chem. 276:27764-9 (July 2001) report that there are three sequence variants in the tandem repeat region of MUC1. Variant 1 replaced DT with ES.

Soares et al., J. Immunol. 166: 6555-63 (June 2001) used a seven tandem repeat MUC1 peptide to elicit an immune response. If the peptide was delivered on dendritic cells, it only elicited T cell immunity. If injected together with soluble peptide, Ab production was also triggered.

Von Mensdorff-Pouilly et al., J. Clin. Oncol. 18:574-83 (February 2000) used a MUC1 triple tandem repeat peptide conjugated to BSA in an immunoassay of anti-MUC1 antibody levels in breast cancer patients.

Denton, et al., Pept. Res. 7:258-64 (September/October 1994), colinearly liked a MUC1 mucin B cell peptide epitope to a known murine T cell epitope in both T-B and B-T orientations. Brossart et al., Blood, 93:4309-17 (June 1999) analyzed the MUC1 amino acid sequence and identified two novel peptides with a high binding probability to the HLA-A2 molecule. One was from the variable tandem repeat region, and the other from outside it.

Carmon, et al., Int. J. Cancer, 85:391-7 (February 2000) evaluated the anti-tumor potential of HLA-A2.1 motif-selected peptides from non-tandem repeat regions of the molecule. See also Pietersz et al., Vaccine, 18:2059-71 (April 2000).

Keil, et al. Angew. Chem. Int. Ed. Engl. 40:366-9(January 2001) conjugated a MUC1 epitope to a tetanus toxin epitope.

Von Mensdorff-Pouilly et al., Int. J. Cancer, 86:702-12 (June 2000) reported that the most frequent minimal epitopic sequences of natural MUC1 IgG and IgM antibodies were RPAPGS (AAs 9-14 of SEQ ID NO:1), PPAHGVT (AAs 17-20 followed by AAs 1-3 of SEQ ID NO:1) and PDTRP (AAs 6-10 of SEQ ID NO:1). MUC1 peptide vaccination induced high titers of IgM and IgG antibodies predominantly directed, respectively, to the PDTRPAP (AAs 6-12 of SEQ ID NO:1) and the STAPPAHGV (AAs 14-20 followed by AAs 1-2 of SEQ ID NO:1) sequences of the tandem repeat. Natural MUC Abs from breast cancer patients reacted more strongly with Galac-glycosylated peptides than with unglycosylated peptides. See also EP Appl 1,182,210; Sandrin, U.S. Pat. No. 6,344,203; Finn, U.S. Pat. No. 5,744,144.

See also, Petrakou, et al., “Epitope Mapping of Anti-MUC1 Mucin protein Core Monoclonal Antibodies” (21-29); Imai, et al., “Epitope Characterization of MUC1 Antibodies” (30-34), Schol, et al., “Epitope Fingerprinting Using Overlapping 20-mer peptides of the MUC1 Tandem repeat sequence” (35-45), and Blockzjil, “Epitope characterization of MUC1 Antibodies” (46-56), all in ISOBM TD-4 International Workshop on Monoclonal Antibodies against MUC1 November 1996), reprinted in Tumor Biology, 19 Suppl. 1:1-152 (1998).

See also Von Mensdorff-Pouilly, et al., “Human MUC1 mucin: a multifacted glycoprotein,” Int J. Biol. Markers, 15:343-56 (2000).

In some embodiments, the present invention therefore contemplates immunogens which comprise at least one native B and/or T cell epitope of MUC1, or at least one mutant epitope substantially identical to such a native epitope. It may further comprise additional MUC1 sequence which is not part of an epitope.

The immunogen may comprise both a B cell epitope and a T cell epitope of MUC1 (which, in each case, may be a natural epitope or an allowed mutant thereof), and these epitopes may be identical, overlapping, or distinct. T and B cell epitopes of an antigen may overlap. For example, in the case of MUC-1, SAPDTRP (AAs 4-10 of SEQ ID NO:1) is a T-cell epitope, while PDTRP (AAs 6-10 of SEQ ID NO:1) is merely a B-cell epitope.

It may further comprise additional B cell epitopes, and/or additional T cell epitopes. The B cell epitopes may be the same or different, and likewise the T cell epitopes may be the same or different.

If the immunogen of the present invention comprises a MUC1-related sequence at least substantially identical to a MUC1 sequence of at least five amino acids, the MUC1-related sequence may comprise one or more glycosylation sites found in the corresponding MUC1 sequence. It may differ from the corresponding MUC1 sequence in the number of potential glycosylation sites, as a result of mutation, or it may have the same number of potential glycosylation sites.

The potential glycosylation sites may be (1) sites actually glycosylated in the MUC1-derived tumor glycoprotein, (2) sites potentially glycosylatable but not actually glycosylated in that tumor glycoprotein, and/or (3) sites foreign to said glycoprotein. Likewise, the actual glycosylation sites may be (1) sites actually glycosylated in the MUC1-derived tumor glycoprotein, (2) sites potentially glycosylatable but not actually glycosylated in that tumor glycoprotein, and/or (3) sites foreign to said glycoprotein. None, one, some or all of the glycosylation sites normally glycosylated in the MUC1-derived tumor glycoprotein may be glycosylated in the immunogen of the present invention.

MUC1 is a polymorphic antigen characterized by a variable number (typically 21-125, especially 41 or 85) of perfect and imperfect repeats of the following sequence:

| | |
|---|---|
| GVTSAPDTRPAPGSTAPPAH | (SEQ ID NO:1) |

Since there are multiple repeats of this sequence, the starting point shown is arbitrary, and an epitope may bridge two repeats.

Consequently, the immunogens of the present invention may comprise the aforementioned complete repeat sequence or a cyclic permutation thereof. Moreover, they may comprise two or more copies of the aforementioned repeat or a cyclic permutation thereof. Thus, in compounds 1a and 1b, there are two copies of a cyclic permutation (starting at TSA . . . and ending with HGV) of the above sequence, followed by the unrelated SSL sequence.

Each MUC1 epitope in question may correspond to an epitope of the variable tandem repeat region, or to an epitope outside that region. The former include RPAPGS (AAs 9-14 of SEQ ID NO:1), PPAHGVT (AAs 4-10 of SEQ ID NO:7) and PDTRP (AAs 6-10 of SEQ ID NO:1). The sequence PDTRPAPGS (AAs 6-14 of SEQ ID NO:1) is of particular interest, as it includes two overlapping epitopes. The PDTRP sequence forms the tip of a protruding knob exposed to solvents and forming a stable type II beta-turn.

The non-VNTR region epitopes include MUC(79-87) (TLAPATEPA) (SEQ ID NO:7), MUC(167-175) (ALGSTAPPV) (SEQ ID NO:8) and MUC(264-72) (FLSFHISNL) (SEQ ID NO:9).

Preferably, the immunogen comprises the polymorphic epitope P[D/E] [T/S]RP or a substantially identical mutant thereof. (“Substantially identical” is defined in Koganty4A-PCT, PCT/US03/10750, filed Apr. 9, 2003.) More preferably it comprises PDTRP or a substantially identical mutant thereof.

In some embodiments, the immunogen comprises at least one 20 amino acid sequence (an effective tandem repeat) which differs solely by one or more conservative substitutions and/or a single nonconservative substitution from a tandem repeat of MUC1, and comprises an epitope of the variable tandem repeat region of MUC1 (either identically, or an allowed mutant). (Conservative substitutions are defined in Koganty4A-PCT, PCT/US03/10750, filed Apr. 9, 2003) Preferably, it differs solely, if at all, by conservative substitutions, more preferably, by no more than a single conservative substitutions, and most preferably, is identical to such a tandem repeat. It should be noted that the tandem repeats of MUC1 are imperfect and hence the sequence could be identical to one repeat but not to another. Also, there are allelic variations in these repeats, and so the sequence could be identical to the sequence for one allele and not for another.

In a subset of these embodiments, the immunogen comprises a plurality of nonoverlapping effective tandem repeats, such as two (for a total of 40 amino acids), three (for a total of 60 amino acids), four, five, six, seven or eight. These effective tandem repeats may, but need not be, identical to each other. (In contrast, note that in the natural human MUC1 mucin, the number of repeats is typically 21-125.)

Besides one or more effective tandem repeats, the peptide portion of the immunogen may comprise additional amino acid subsequences. If so, these subsequences may comprise additional epitopes, which may be MUC1 variable tandem repeat region epitopes (falling short of a effective tandem repeat), MUC1 epitopes from outside that region, or epitopes of other cancer antigens. It may also include an immunomodulatory element, see Longenecker5-PCT, PCT/US95/04540, filed Apr. 12, 1995.

Preferably, one or more of the serines and/or threonines of the MUC1 tandem repeat are glycosylated, preferably with Tn or sialyl Tn. In the natural human MUC1 mucin, there are five normal glycosylation sites per repeat. In normal MUC1, an average of 2.6 of these five sites are in fact occupied. The average number of glycosylated amino acids per repeat may be less than, the same as, or greater than the “natural” value.

### Other Breast Cancer Associated Epitopes

At least one immunogen may comprise at least one epitope of another breast cancer-associated antigen, including but not limited to:
MAGE family, e.g. MAGE-1,2,3, and 6
NY-ESO-1
HER-2
P53
Kinesin 2
NY-BR-1
Mucins, e.g. MUC-1, and 5
Telomerase
CYFRA 21-1 (cytokeratin fragment 19)
ART 1
CEA
SSX family, e.g. SSX-1,2, and 4
SCP-1 (synaptonemal complex protein 1)

### Clustered Epitopes

If the immunogen comprises a plurality of epitopes, they may be clustered or unclustered. A cluster is here defined as a moiety consisting of at least two directly adjacent epitopes. The cluster may of course include more than two epitopes in direct sequence. See, e.g., Reddish, et al., Glycoconjugate J., 14:549-60 (1997) (clustered STn), Ragapathi, et al. Cancer Immunol. Immunother. 48:1-8 (1999).

### Immunogen Design

A natural or non-natural immunogen may comprise one or more epitopes and, if it comprises a plurality of epitopes, they may be the same or different. The epitopes may be clustered or unclustered.

### Hapten-Carrier Conjugate

If the epitopes are B cell epitopes, then the immunogen must be of sufficient size to elicit a humoral immune response.

In a first preferred design, the epitope in question is conjugated to an unrelated (non-breast cancer associated) immunogenic carrier, such as KLH, albumin, dextran, etc. The immunogenic carrier is a chemical moiety which does not itself comprise any of the desired epitopes but which, suitably conjugated to one or more such epitopes, creates an immunogenic conjugate which elicits a humoral immune response. Typically, the conjugate will have a molecular weight of at least 5,000 daltons, more preferably at least 10,000 daltons. The preferred maximum is the maximum exhibited by mucins, e.g., about 5,000,000 daltons.

If the epitope and the carrier are both peptides, then the conjugate may be expressed directly by recombinant DNA technique, i.e., an artificial gene encodes the entire conjugate. If so, then the number of epitope-encoding segments within the artificial gene will dictate the number of epitopes within a single conjugate molecule.

Otherwise, the conjugate must be chemically synthesized. Usually, the epitope and carrier are prepared separately and then conjugated chemically. If so, the number of epitopes conjugated to a single carrier moiety within a given conjugate molecule will vary from molecule to molecule. The maximum substitution ratio (# epitopes per conjugate molecule) will be determined by the number of reactive functionalities on the carrier. For example, if the carrier is a protein, and the conjugation chemistry requires an amino function on the carrier, then the maximum substitution ratio will be the number of lysines, plus one (for the N-terminal).

Many workers have synthesized glycosides of the carbohydrate haptens and of their sialylated analogs and have used these glycosides to conjugate the haptens to proteins or synthetic peptide carriers. The glycosides have generally included an aglycon moiety from which a highly reactive functionality can be generated without altering the saccharide portion of the respective hapten glycoside. The “activated” hapten glycosides are then reacted with amino groups of the proteins or synthetic peptide carriers to form amide of Schiff base linkages. The Schiff base grouping can be stabilized by reduction with a borohydride to form secondary amine linkages; the whole coupling process is then referred to as reductive amination. (Gray, Arch. Biochem. Biophys., 163, 426, 1974). For examples of these conjugates, see Lemieux, et at., U.S. Pat. No. 4,866,045; Naicker, et al., U.S. Pat. No. 4,935,503; Kolar, U.S. Pat. No. 4,42_{—}284; Feizi, U.S. Pat. No. 4,563,445; Koganty, U.S. Pat. No. 5,055,562; Jennings, U.S. Pat. No. 4,356,170; Roy, U.S. Pat. No. 5,034,516, Wong, U.S. Pat. No. 6,013,779.

### Linear Multimer Design

It is possible to incorporate a sufficient number of epitopes into a linear immunogen so that a distinct immunogenic carrier moiety is unnecessary, i.e., the epitopes collectively act as an immunogenic carrier moiety for each other. In this design, a plurality of epitopes, which may be the same or different, are conjugated together, either directly, or with the aid of short spacer moieties. Typically, this kind of immunogen comprises 3-30 epitopes.

The linear immunogen in question can be prepared by recombinant DNA techniques if the epitopes are all peptide epitopes.

It is also possible to prepare an intermediate, containing any desired peptide epitopes, by recombinant DNA techniques, and then glycosylate the intermediate so as to add on the desired carbohydrate epitopes.

### Crosslinked Multimer Design

It is also possible to prepare smaller linear multimers and then crosslink them together.

### Dendrimer Design

In another design, the immunogen has a branched structure, with one or more epitopes attached to each of a plurality, and preferably to all, of the branches. For example, the epitopes may be attached to a branched lysine core structure. Each lysine can conjugate to three other lysines by virtue of the normal N-terminal amino group, the normal C-terminal carboxy group, and the epsilon amino group on its side chain. It should be noted that this dendrimer design usually differs from the conventional hapten-carrier conjugate in that the core (the carrier) is branched, not linear, and in that the core is a relatively small portion of the entire conjugate, e.g., less than half of the molecular weight of the conjugate.

This recitation of possible immunogen designs is not intended to be exhaustive.

### Protein Carrier

The preferred protein carrier, if any, in the immunogens of the present invention is a macromolecule with, in monomeric form, a molecular weight of at least 10 kD, and which contains one or more lysine residues. Preferably, it is at least 3% lysine (by moles).

The preferred protein carrier is a hemocyanin, such as an arthropod or molluscan hemocyanin. Hemocyanins of gastropods, especially of the Fissurellidae (keyhole limpets), and in particular the keyhole limpet (Megathura crenulata) hemocyanin, are most preferred.

Hemocyanins are the oxygen transport proteins of many arthropods and molluscs. Keyhole limpet hemocyanin, in nature, is a multimer, with a total MW of about 8,000 kDa. The monomer is about 400 kDa. It consists of two immunologically and physiologically distinct isoforms, KLH1 and KLH2. Both are present in the hemolymph as cylindrical didecamers. Each isoform monomer contains eight functional units (FUs), termed “a” to “h” from the N- to C-terminal. FUs “b” to “g” of KLH1 total 2141 a.a., and FUs “b” to “h” of KLH2 total 2473 a.a. See Altenheim, et al. “Sequence of Keyhole Limpet Hemocyanin”, Abstract, http://www.sb-roscoff.fr/IO2BiP/I02B1PP.PDF; Swerdlow, Comp. Biochem. Biophys. 113B:537-48 (1996); Stoeva, Biochem. Biophys. Acta 1435:94-109 (1999); Harris and Markl, Micron., 30:597-623 (1999). Swerdlow reports that KLH-A is 449 kDa and KLH-B is 392 kDa. Sohngen, et al., Eur. J. Biochem., 248:602-14 91997) reports that KLH1 is 400 kDa and KLH2 is 345 kDa. Ebert, U.S. Pat. No. 5,855,919 uses the value of 400 kDa.

Preferably, in the conjugates of the present invention, if the monomeric unit of the carrier moiety is the KLH monomer, the conjugate is not a substituted decamer, didecamer or multidecamer of the KLH monomer.

The native KLH is rich in copper, but copper is lost during reductive amination. KLH is glycosylated, with a carbohydrate content of about 4% of molecular mass. See Harris, supra.

Preferably, it KLH monomer is used, at least one carbohydrate hapten moiety will be one not natively associated with KLH. Preferably, at least one component sugar of the carbohydrate hapten moieties will be one not natively associated with KLH.

### Aggregated Multimeric Protein Carrier

In a preferred embodiment, the epitopes are conjugated to an aggregated multimeric protein carrier, in particular, aggregated KLH. (Note that here the term “multimeric” refers to the number of copies of the carrier moiety.)

The aggregation results from the interaction of individual monomers of the protein carrier to form a multimeric entity. The interaction may be through binding, and/or through entanglement of the individual protein chains (before, during or after attachment of the carbohydrate haptens). If binding contributes to the oligomerization, it may be covalent and/or noncovalent.

Preferably, the aggregation occurs more or less simultaneously with the attachment of the carbohydrate haptens to the protein.

The multimeric entities preferably are dimers, trimer, tetramers, and/or pentamers of the monomeric unit of the protein carrier.

It is believed that the immunogenic potency of these preparations is attributable to the combination of a high hapten substitution ratio, and the aggregation of the protein carriers to form multimeric entities.

### Degree of Aggregation

The preferred immunogen of the present invention is an aggregated, carbohydrate hapten-substituted KLH. Each monomeric unit may be KLH1, KLH2 or some other KLH monomer.

The term “substituted KLH monomer”, as used herein, means KLH substituted with a plurality of carbohydrate haptens in addition to those with which it is natively associated. These could be duplicates of existing native carbohydrate chains, but more usually will include haptens not natively associated with KLH. The KLH may, but need not, be deglycosylated to remove some or all of the native carbohydrate, specifically or nonspecifically, before hapten substitution.

The molecular weight of the substituted KLH monomer will be greater than that of the unsubstituted KLH. If the latter is 400 kDa (literature values range from 345 to 449 kDa), then the substituted KLH will be of greater MW. The increment will depend on the molecular weight of each hapten moiety (including the linker) and on the number of hapten moieties per monomer.

If the unsubstituted KLH monomer is 400 kDa, then a substituted dimer necessarily has molecular weight greater than 800 kDa. Hence, the substituted aggregate preferably has an apparent molecular weight of more than 800 kDa, more preferably more than 1,200 kDa, still more preferably more than 1,600 kDa.

KLH in the preferred carbohydrate hapten-substituted monomeric form has a molecular weight of about 500 KD; without the added carbohydrate (and linkers), it is about 400 kD. Thus, hapten substitution may increase molecular weight by 25%, or more, relative to the unsubstituted KLH monomer. It follows that the substituted aggregate also preferably has an apparent molecular weight of at least 1,000 KDa, more preferably at least 1,500 KDa, even more preferably at least 2,000 KDa.

It will be understood that the preparation may comprise a heterogeneous mixture of n-mers, e.g., monomers, dimers, trimers, tetramers, etc., so that the apparent molecular weight is actually the weighted average of the apparent molecular weight of each size class of n-mer.

The preparation could theoretically be fractionated by molecular weight to determine the fraction attributable to each size class of n-mer. Preferably, monomers are less than 50% (by weight) of the preparation, more preferably less than 25%, still more preferably less than 10%, most preferably less than 5%. The preparation may also be fractionated with the goal of discarding the predominantly monomeric fraction(s) and thereby enriching for multimers.

The maximum limit on the degree of aggregation is that the aggregate should not be so large as to precipitate out of solution. However, the apparent molecular weight is preferably less than 5,000 kDa (equivalent to a substituted decamer), and more preferably less than 2,500 kDa (equivalent to a substituted pentamer).

Apparent molecular weight is preferably determined by laser light scattering. See Wyatt, Anal. Chim. Acta, 272:1-40 (1993). It may be estimated by size exclusion (molecular sieve) chromatography, as set forth in Krantz2.1-PCT, PCT/US02/24735, filed Aug. 5, 2002.

Preferably, the aggregated immunogen of the present invention has a potency which is at least 200% of that of a conjugate of the same hapten and carrier, in the same hapten carrier monomer substitution ratio, wherein the carrier is unaggregated. For this purpose, potency is measured by the antibody response of immunized mice.

### STN-KLH Conjugates

The most preferred immunogen of the present invention is an immunogen comprising STn, preferably as a conjugate to KLH, and most desirably similar or identical to the immunogen incorporated into Biomira's Theratope® vaccine.

Preferably, the KLH carrier is aggregated, so that each conjugate molecule comprises, on average, at least a KLH dimer, more preferably a KLH trimer, and still more preferably a KLH tetramer.

Preferably, the NANA content is greater than 3%, more preferably at least 5%, such as about 7%.

The preferred immunotherapeutic compound of this invention can be made according to International Application Number PCT/US02/24735 (International Publication Number WO03015796). See also International Application number PCT/US90/01856 (International Publication Number WO9011764).

### Conjugation

In the case of the chemical conjugates, one or more hapten molecules are conjugated to each carrier molecule. The point of attachment on the carrier is ordinarily an accessible amino group, such as the amino terminal of the carrier, or more usually the epsilon amino group of lysine.

The hapten is conjugated to this point of attachment, either directly, or through a linker. Usually, the linker is not a carbohydrate or peptide itself. The linker, if any, is preferably a small aliphatic group consisting of carbon, hydrogen, and optionally, oxygen, nitrogen and/or sulfur, of not more than 12 atoms other than hydrogen. More preferably it is an alkyl group, linear or branched, of not more than 12 carbon atoms. Even more preferably it is —(CH₂)ₙ where n=1 to 12. Most preferably it is —CH₂CH₂—. Each linker will connect an oxygen of the carbohydrate hapten to an amino nitrogen, i.e., the epsilon nitrogen of lysine, or the amino terminal of the protein carrier.

The linker may be bifunctional (attaching just one hapten to the carrier monomer) or polyfuntional (in which case one linker may attach a plurality of haptens to the carrier monomer).

A “linking agent” is reacted with A and B to form the structure A-linker-B, the “linker” being related in structure to the original linking agent. The reaction may be simultaneous, or the linking agent may be reacted first with A to form the structure A-linking arm, and then the latter with B to form A-linker-B.

If the hapten-linking arm is hapten-crotyl (e.g., STn-crotyl), then ozonolysis generates a reactive hapten aldehyde, which can be used in reductive amination of the carrier to yield hapten-CH₂CH₂-carrier, i.e., the preferred two-carbon linker. The hapten is usually O-linked to the linker, but other linkages are possible.

Another linking agent of interest is an MMCCH linking agent, 4-(4-maleimidomethyl)cyclohexane-1-carboxyl hydrazide. See Ragaputhi, et al., Cancer Immunol. Immunother. 48:1-8 (1999).

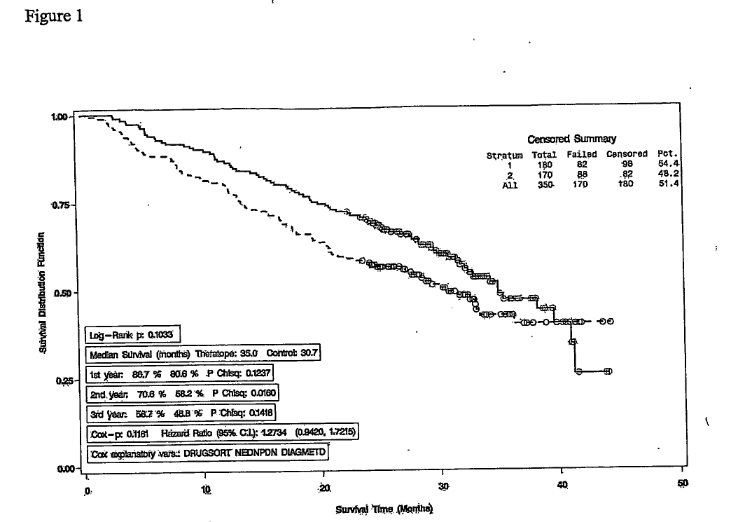

Some polyfunctional linkers based on crotyl linking chemistry are depicted in FIG. 1 of Reddish, et al., Glycoconjugate J., 14:549-60 (1997).

Methods of carrying out conjugation reactions and, in particular, of preparing an Stn-KLH conjugate in which the KLH is aggregated, are further described in Krantz2.1-PCT, PCT/US02/24735, filed Aug. 5, 2002.

### Hapten-Substitution Ratio

Preferably, the ratio of hapten to carrier is at least 10 molecules of conjugated hapten to each carrier monomer. The maximum ratio is determined by the number of accessible attachment sites. Usually, the ratio is in the range of 10-120.

In the case of sialylated conjugate, such as an STn/KLH conjugate, the NANA content is indicative of the hapten substitution ratio (the number of sialylated haptens per carrier monomer). NANA content may be assayed as set forth below.

In the case of an STn/KLH conjugate, the NANA content is preferably in excess of 3%, more preferably at least 4%, still more preferably at least 5%, even more preferably at least 6%, most preferably at least 7%. Preferred values include those values in excess of 3% which are set forth in Tables 1 and 2.

The maximum possible NANA content is a function of the total number of possible STn attachment sites on the KLH. Assuming that an STn is attached to every lysine side chain of KLH, the NANA content would be about 12% by weight of conjugate. This does not include the molecular weight of the linker or the Tn. If calculated relative to the molecular weight of the unsubstituted KLH, it would be about 13%. The whole hapten-linker arm content would be about 19% relative to the molecular weight of the unsubstituted KLH.

Higher NANA content is primarily achieved by increasing the ratio of hapten-to-KLH in the glycosylation reaction.

If the amount of hapten is increased to elevate NANA content, but this does not result in an increase in immunogenicity, then the extra hapten is, in effect, wasted. Hence, it may be desirable to limit the NANA content, for economic reasons, to be not more than 10% by weight.

Thus, the NANA content of an STn-KLH conjugate is most preferably in the range of 6 to 10% by weight.

### Immunomodulators

The immunogens of the present invention, which comprise breast cancer associated epitopes, may be used in conjunction with substances which, while not such immunogens, modulate the immune response to such immunogens in a desirable manner.

Adjuvants

The immunogens of the present invention may be used in conjunction with any known adjuvant. The adjuvant may be chemical or microbial in nature. Possible adjuvants include Enhanzyn™, Lipid-A, CFA, SAF-1, MDPI BCG, liposomes, and Bordetella pertussis toxin, and their derivatives and analogues. Enhanzyn™ is preferred, but the present invention is not limited to the use of any particular adjuvant. The tumor-associated hapten may be conjugated to other carrier proteins, such as tetanus or diphtheria toxoid, or retrovirus peptides (e.g., VP6 viral peptide), rather than KLH, and the hapten/molecule-to-carrier molecule substitution ratio may be varied. Either natural or synthetic antigens which cross-react with the immunosuppressive mucin may be employed.

Cyclophosphamide

The immunogens of the present invention may be used in conjunction with an immunopotentiating amount of cyclophosphamide. Cyclophosphamide (N,N-bis[2-cholorethyl]tetrahydro-2H-1,3,2-oxazaphosphorine-2-amine-2-oxide), a nitrogen mustard derivative, is a cytotoxic agent which causes cross-linking of DNA. It is most effective against rapidly dividing cells, hence its use in cancer chemotherapy. Since it also destroys lymphocyte cells, it is also useful as a immunosuppressive agent, indeed, it is one of the most potent immunodepressants known.

Although most chemotherapeutic agents suppress host immunity, it has been demonstrated that certain chemotherapeutic agents, under specific conditions, are able to augment host anti-tumor immunity. It is believed that this can arise because some tumors express mucins which elicit suppressor T cell activity, and it is that response whose inhibition is desirable.

The time interval between administration of the cyclophosphamide and administration of the synthetic tumor-associated glycoconjugate is not fixed, but is dependent on the time of onset and duration of action of the cyclophosphamide's inhibitory effect on suppressor T cell activity or on the induction of such activity by tumor-expressed mucins. The dosage of cyclophosphamide may be selected to increase the antigenic specificity of the anti-suppressor T cell activity effect.

In place of cyclophosphamide, another antagonist of immunosuppression may be employed, such as other oxazaphosphorines, cimetidine or an anti-(suppressor cell) or anti-(suppressor factor) monoclonal antibody. Numerous antibodies of these two types are offered for sale (see Linscott's Directory of Immunological and Biological Reagents, p. 10, 5th ed., 1988,89).

The present invention is not to be restricted on the basis of the present interpretation of the mechanism whereby cyclophosphamide or a similar agent exercises an immunopotentiating effect. An agent antagonizes the immunosuppressive effect of a tumor-associated mucin if it interacts with the mucin or the T cell so that the mucin no longer activates suppressor T cell activity, or if it interacts with a T cell so activated or its suppressor factors so as to diminish the suppressor activity induced by said mucin, or if it interacts with other components of the cellular immune system so as to render them less vulnerable to suppressor T cells activated by said mucin or to suppressor factors released by such cells, in another embodiment, a monoclonal antibody specific for an epitope of a tumor-associated, immunosuppressive mucin is attached to a suitable support to form an immunosorbent. Circulating tumor-associable immunosuppressive mucins recognized by the immunosorbent are removed from the patient's bloodstream by plasmapheresis. The immune response to the tumor, with or without further stimulating the immune system by active specific tumor immunotherapy, is thereby enhanced. (Lectins or other binding substances might be-used in place of antibodies).

### Progestins and Anti-Progestins

Progestins (such as progesterone) are another type of hormone that play a role in the normal development of breast tissue. The evidence as to the effect of progrestins on the development of breast cancer is mixed. Both progestins and anti-progestins have been proposed for use in the treatment of breast cancer. To the extent that administration of a progestin or an anti-progestin can have a protective effect against breast cancer, its use is within the contemplation of the present invention. Mifepristone (RU 486) is an example of an anti-progestin.

### Chemotherapy

In addition to anti-hormonal (anti-estrogenic steroid) therapy and immunotherapy, other chemotherapy may be employed. Anthracyclines (for example: doxorubicin, daunorubicin, epirubicin, idarubicin) may be used in the treatment of metastatic breast cancer. Several mechanisms of action may play a role in the anti-tumour effects of anthracyclins, and include: intercalation of DNA, interaction with topoisotnerase II, causing strand breaks (single and double) in DNA and formation of free radicals.

Taxanes (for example: paclitaxel, docetaxel) may also be used in the treatment of metastatic breast cancer. Taxanes are antimicrotubular compounds that inhibit cell division by binding to tubulin and inhibiting microtubular disassembly that is required for cell division.

### Use in Conjunction (Combination)

When two therapies are used in conjunction (or combination), it means that both therapies are employed, but it does not mean that they necessarily are administered simultaneously. Rather, it means that one therapy is used while the other therapy is still exercising an effect on the patient. Preferably, they are used at such times as to have a synergistic effect, that is, the combined effects are greater than those which would be reasonably expected as the simple additive effect of the individual therapies.

### Therapeutically Effective

A therapy, or combination of therapies, is therapeutically effective if there is a statistically significant (p=0.05) improvement in the therapeutic outcome in the treatment group as compared to a control group. It is not required that the therapy cure or even merely alleviate the condition of all treated patients.

When a combination therapy is contemplated, the individual agents may be used in amounts which individually would not be therapeutically effective, if they would be effective in combination.

### Immunogenic Compositions

The immunogens of the present invention may be administered as a component of an immunogenic composition. Immunogenic compositions are compositions which comprise, as at least one immunological agent, an immunogen.

The immunogenic composition may further comprise a liposome. Preferred liposomes include those identified in Jiang, et al., PCT/US00/31281, filed Nov. 15, 2000 (our docket JIANG3A-PCT), and Longenecker, et al., 08/229,606, filed Apr. 12, 1994 (our docket LONGENECKER5-USA, and PCT/US95/04540, filed Apr. 12, 1995 (our docket LONGENECKER5-PCT). A variety of methods are available for preparing liposomes, as described in, e.g., Szoka et al., Ann. Rev. Biophys. Bioeng. 9:467 (1980), U.S. Pat. Nos. 4,235,871, 4,501,728, 4,837,028, and 5,019,369, incorporated herein by reference.

The composition may further comprise antigen-presenting cells, and in this case the immunogen may be pulsed onto the cells, prior to administration, for more effective presentation.

The composition may contain auxiliary agents or excipients which are known in the art. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Katzung, ed. Basic and Clinical Pharmacology, Fifth Edition, Appleton and Lange, Norwalk, Conn. (1992), which references and references cited therein, are entirely incorporated herein by reference.

A composition may further comprise an adjuvant to nonspecifically enhance the immune response. Some adjuvants potentiate both humoral and cellular immune response, and other s are specific to one or the other. Some will potentiate one and inhibit the other. The choice of adjuvant is therefore dependent on the immune response desired.

A composition may include immunomodulators, such as cytokines which favor or inhibit either a cellular or a humoral immune response, or inhibitory antibodies against such cytokines.

### Pharmaceutical Purposes

A purpose of the invention is to protect subjects against a disease. The term “protection”, as in “protection from infection or disease”, as used herein, encompasses “prevention,” “suppression” or “treatment.” “Prevention” involves administration of a Pharmaceutical composition prior to the induction of the disease. “Suppression” involves administration of the composition prior to the clinical appearance of the disease. “Treatment” involves administration of the protective composition after the appearance of the disease. Treatment may be ameliorative or curative.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “protection,” as used herein, is meant to include “prophylaxis.” See, e.g., Berker, supra, Goodman, supra, Avery, supra and Katzung, supra, which are entirely incorporated herein by reference, including all references cited therein.

The “protection” provided need not be absolute, i.e., the disease need not be totally prevented or eradicated, provided that there is a statistically significant improvement (p=0.05) relative to a control population. Protection may be limited to mitigating the severity or rapidity of onset of symptoms of the disease. An agent which provides protection to a lesser degree than do competitive agents may still be of value if the other agents are ineffective for a particular individual, if it can be used in combination with other agents to enhance the level of protection, or if it is safer than competitive agents.

The effectiveness of a treatment can be determined by comparing the duration, severity, etc. of the disease post-treatment with that in an untreated control group, preferably matched in terms of the disease stage.

The effectiveness of a prophylaxis will normally be ascertained by comparing the incidence of the disease in the treatment group with the incidence of the disease in a control group, where the treatment and control groups were considered to be of equal risk, or where a correction has been made for expected differences in risk.

In general, prophylaxis will be rendered to those considered to be at higher risk for the disease by virtue of family history, prior personal medical history, or elevated exposure to the causative agent.

### Methods of Administration

The therapeutic agents of the present invention may be administered by any effective route of administration, including oral or parenteral, the latter including intravenous, subcutaneous, and intramuscular.

### Dosage Forms

The therapeutic agents of the present invention may be administered in any effective dosage form, including tablets, capsules, injectants, and so forth.

The immunogen may be delivered in a manner which enhance, e.g., delivering the antigenic material into the intracellular compartment such that the “endogenous pathway” of antigen presentation occurs. For example, the immunogen may be entrapped by a liposome (which fuses with the cell), or incorporated into the coat protein of a viral vector (which infects the cell).

Another approach, applicable when the immunogen is a peptide, is to inject naked DNA encoding the immunogen into the host, intramuscularly. The DNA is internalized and expressed.

It is also possible to prime autologous PBLs with the compositions of the present invention, confirm that the PBLs have manifested the desired response, and then administer the PBLs, or a subset thereof, to the subject.

### Dosage Schedule

The present invention contemplates the use of a combination of agents, and these may have different dosage schedules.

It is understood that the effective dosage will be dependent upon the age, sex, health, and weight of the recipient, kind of concurrent treatment, if any, frequency of treatment, and the nature of the effect desired. The ranges of effective doses provided below are not intended to limit the invention and represent preferred dose ranges. However, the most preferred dosage will be tailored to the individual subject, as is understood and determinable by one of skill in the art, without undue experimentation. This will typically involve adjustment of a standard dose, e.g., reduction of the dose if the patient has a low body weight. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Chabner et al., supra; De Vita et al., supra; Salmon, supra; Schroeder et al., supra; Sartorelli et al., supra; and Katsung, supra, which references and references cited therein, are entirely incorporated herein by reference.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects. See, e.g., Berkow, et al., eds., The Merck Manual, 15th edition, Merck and Co., Rahway, N.J., 1987; Goodman, et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985), which references and references cited therein, are entirely incorporated herein by reference.

The total dose required for each treatment may be administered in multiple doses (which may be the same or different) or in a single dose, according to an immunization schedule, which may be predetermined or ad hoc. The schedule is selected so as to be therapeutically effective, i.e., to provide protection. The doses adequate to accomplish this are defined as “therapeutically effective doses.” (Note that a schedule may be therapeutically effective even though an individual dose, if administered by itself, would not be effective, and the meaning of “therapeutically effective dose” is best interpreted in the context of the immunization schedule.)

Amounts effective for this use will depend on, e.g., the agent, the manner of administration, the stage and severity of the disease being treated, the weight and general state of health of the patient, and the judgment of the prescribing physician.

Typically, the daily dose of an active ingredient of a pharmaceutical, for a 70 kg adult human, is in the range of 10 nanograms to 10 grams. For immunogens, a more typical daily dose for such a patient is in the range of 10 nanograms to 10 milligrams, more likely 1 microgram to 10 milligrams. However, the invention is not limited to these dosage ranges.

The doses may be given at any intervals which are effective. In the case of immunogens, if the interval is too short, immunoparalysis or other adverse effects can occur. If the interval is too long, immunity may suffer. The optimum interval may be longer if the individual doses are larger. Typical intervals are 1 week, 2 weeks, 4 weeks (or one month), 6 weeks, 8 weeks (or two months) and one year. The appropriateness of administering additional doses, and of increasing or decreasing the interval, may be reevaluated on a continuing basis, in view of the patient's immunocompetence (e.g., the level of antibodies to relevant antigens).

### EXAMPLES

Background: THERATOPE® vaccine is an investigational therapeutic cancer vaccine consisting of a synthetic form of the tumor associated antigen Sialyl Tn (STn) conjugated to the aggregated carrier protein keyhole limpet hemocyanin (KLH), at a hapten substitution ratio yielding a NANA content of about 7%.

Patients and Methods: Metastatic breast cancer patients (MBC pts) who had no evidence of disease (NED) or non-progressive disease (NPD) following any first-line chemotherapy were randomized 1:1 to receive adjuvant plus THERATOPE® vaccine or control [adjuvant plus KLH]. All patients received a single, low-dose, IV infusion of cyclophosphamide before vaccine. Primary endpoints were time to disease progression (TDP) and overall survival (OS). Pts were stratified by disease status and concomitant hormone therapy (HT).

### Introduction

An international phase III clinical trial comparing Theratope® vaccine (STn-KLH) to control vaccine (KLH alone) in 1028 women with stable or responding metastatic breast cancer following any first line chemotherapy, was conducted. The primary efficacy endpoints were survival and time to disease progression (TDP). The two patient treatment groups were well balanced in terms of prognostic characteristics including age, estrogen receptor (ER) status, progesterone receptor (PR) status, HER2 expression, time from primary diagnosis to first metastasis, disease burden, response to chemotherapy, and concomitant hormone therapy use.

While the trial yielded disappointing efficacy results in terms of the overall intent-to-treat (ITT) population (survival Cox p=0.916 and TDP Cox p=0.353), encouraging results were observed in the subgroup of patients who received THERATOPE® vaccine concomitantly with anti-hormone (anti-estrogenic steroid) therapy. This therapy was with SERMs and/or aromatase inhibitors, with the precise anti-hormone therapy varying from patient to patient. The benefit was observable for both the SERM subpopulation and the aromatase inhibitor subpopulation.

The benefit was particularly striking for patients in this subgroup who also developed an immune response to THERATOPE® vaccine at or higher than the median response. This report will discuss in detail the findings of these exploratory analyses, as well as describe the survival outcomes following updates.

### Efficacy Outcomes by ITT Strata

The original protocol stratified patients by response to chemotherapy into no evidence of disease (NED) or non-progressive disease (NPD). Of the 1028 women randomized into the trial, approximately 150 patients were enrolled under this original protocol. In an effort to increase the rate of enrollment, an amendment was approved at each trial site that allowed the enrollment of women who were receiving concomitant anti-tumor hormone therapy for treatment of their metastatic breast cancer, an exclusion criterion in the original protocol. To minimize the potential for bias, it was decided to stratify patients according to this hormone use, in addition to response to chemotherapy, at the time of study entry. The primary analyses utilized the Cox Proportional Hazards model, which included time from diagnosis to first metastasis, as well as the two stratification variables, as explanatory variables.

At the time of the final analysis, TDP and survival were calculated for each of the strata. The results of these calculations are shown in Table 1.

Both TDP and survival advantages were observed for patients in the non-progressive disease strata who received hormone therapy concomitantly with THERATOPE® vaccine (n=152), compared to those in the same strata who received KLH control (n=143). Additionally, patients receiving KLH control who had no evidence of disease and did not receive concomitant hormone therapy (n=38) appeared to do better in terms of survival, but not TDP, than those in the comparable THERATOPE® vaccine arm (n=40). None of the differences between treatment groups in any strata combination achieved statistical significance.

### Efficacy Outcomes by ITT Hormone Groups

The total numbers of patients with non-progressive disease formed a large majority when compared to the summation of the NED patients (916 versus 112 patients, respectively). When the patients were examined by hormone status, approximately one third were stratified to concomitant hormone therapy compared to no hormone therapy (329 versus 699 patients respectively). Although stratification by hormone use was not equally balanced compared to no hormone use, the numbers of patients in each group were large enough to justify exploratory statistical comparisons. These data are shown in Table 2.

When examining the data by hormone group, patients from either treatment group who did not receive concomitant hormone therapy were observed to have a similar median survival and TDP. However, the median survival of patients receiving THERATOPE® vaccine concomitantly with hormone therapy was 4.2 months longer than that of patients receiving KLH control concomitantly with hormone therapy. With regards to TDP, a 2.5-month benefit for THERATOPE® vaccine treated patients receiving concomitant hormone therapy was observed compared to patients in the relevant comparison group receiving KLH control. Although the benefit for THERATOPE® vaccine treated patients in the hormone subset did not reach statistical significance, the results were clinically compelling, and warranted further investigation.

### Antibody Response

THERATOPE® vaccine is designed to stimulate an immune response to the tumor-associated antigen, sialyl Tn (STn). This immune response potentially results in a therapeutic benefit. As a secondary objective, immune response was included in the phase III trial. Sera from patients in this trial were assayed for IgG and IgM antibody responses to STn, and additionally to KLH, and Ovine Submaxillary Mucin (OSM, a naturally occurring monomeric and clustered STn).

Serum assays were performed at baseline, week 12 (following receipt of four adjuvanted vaccinations), week 24 and then at 3-month intervals until documented disease progression. Serum from 364 THERATOPE® vaccine treated, and 323 KLH control treated patients, was available for the week 12 assessment of antibody response. Assay results by treatment arm for all patients and by hormone group (H=hormone, NH=no hormone) are shown in Table 3.

The lower response to KLH in the THERATOPE® vaccine patients may be due to the fact that STn clusters on THERATOPE® vaccine block some of the epitopes on the KLH; therefore THERATOPE® vaccine treated patients had reduced exposure to KLH.

### Survival and TDP by Antibody Response for All Patients

In phase II trials, the magnitude of the IgG anti-OSM titre was associated with improved survival⁽¹⁾. Therefore, exploratory analyses were conducted to assess whether a correlation with response at week 12 and survival would be observed utilizing data from the phase III trial. No statistically significant survival differences were observed in relation to IgM responses. The survival by median IgG antibody response to each of the antigens is shown in Table 4.

For THERATOPE® vaccine treated patients, a 7.2-month survival advantage was observed for those with an IgG OSM titre at or greater than the median, compared to those with a response below the median, with a trend toward statistical significance. A titre at or higher than the median to STn and KLH was also associated with improved survival, but in neither case was the improvement statistically meaningful. No statistically significant differences in TDP were observed based on antibody response to any of the three antigens tested.

A different pattern emerged for the KLH control patients. Both the survival and TDP of these patients was longer for those whose IgG anti-KLH response was less than the median compared to patients with responses at or higher than the median. The rationale for these outcomes are not understood, however, several prognostic variables appeared to favor those patients who developed the lower antibody response to KLH, and may have contributed to the better survival and TDP observed in this group. These include a greater number of ER+ versus ER− women (68% versus 59%), a greater number of superficial or bone only versus visceral metastases (26% versus 16.6%), and a greater number of women with normal versus elevated CA 27.29 at baseline (57.1% versus 51%).

### Survival by Antibody Response for the Hormone Subset

Survival by antibody response was also examined within the subset of patients who received concomitant anti-cancer hormone therapy. Although this subset had a better overall survival outcome compared to patients who did not receive concomitant hormone therapy while on study, the difference was particularly striking for THERATOPE® vaccine treated patients who had an anti-OSM IgG response at or above the median, and for KLH control patients who had an anti-KLH IgG response below the median. The difference in survival by response to STn and KLH in the THERATOPE® vaccine treated hormone subset was not statistically significant. The data are shown in Table 5.

### Survival by Antibody Response for the Non-Hormone Subset

Survival by antibody response was also examined for those patients who did not receive concomitant hormone therapy. Antibody data was available from 23i THERATOPE® vaccine and 206 KLH control treated patients.

For patients who received THERATOPE® vaccine without concomitant hormone therapy, no significant differences in survival were observed related to antibody response to any of the antigens tested. Median survival by a-OSM response was 25.1 months for patients with responses at or above median and 23.7 for patients with responses below median (Cox p=0.8065); median survival by a-STn response was 24.0 months for patients with responses at or above median, and 24.3 for those with responses below median (Cox p=0.9347); median survival by a-KLH response was 26.1 months for patients with responses at or above median and 22.8 for patients with responses below median (Cox p=0.2580).

For the KLH control patients, a statistically significant survival advantage was observed for patients with an IgG anti-KLH response below the median compared to those with responses at or higher than the median (30.2 versus 22.3 months respectively, Cox p=0.0215).

### ITT Overall Baseline CA 27.29 Survival Impact

A baseline serum CA 27.29 level was assessed for patients in both treatment groups (THERATOPE® vaccine and KLH control) utilizing the TRUQUANT BR radioimmunoassay kit (Fujirebio Diagnostics Inc.). CA 27.29 is a tumor-associated antigen correlated with disease stage in breast cancer patients, and has been used as a signal of disease progression or recurrence^{(2,3,4)}.

A total of 52% of the THERATOPE® vaccine treated patients and 50% of KLH control patients had elevated levels of CA 27.29 at baseline. Patients in both arms of the trial with normal CA 27.29 experienced a clear survival advantage over their counterparts with elevated levels.

In the THERATOPE® vaccine treated group, sera from 515 patients were assayed. Of these, 249 patients had CA 27.29 levels within the normal range, and 266 had elevated levels. Their median survival was 28.7 and 19.2 months respectively (Cox p=<0.0001).

In the KLH control group, sera from 500 patients were assayed, with 250 patients in each of the normal and elevated level groups. Patients with a normal CA 27.29 level survived a median 26.6 months, compared to 18.1 months in patients with elevated levels (Cox p=0.0002).

### ITT Overall Week 12 CA 27.29 Relative to Baseline

A total of 270 THERATOPE® vaccine and 231 KLH control patients also had CA 27.29 levels measured at week 12, following receipt of four adjuvanted vaccinations. The data was examined to elicit the number of patients in each treatment group with normal or elevated CA 27.29 at week 12. A further examination of the data was conducted to determine the number of patients who experienced a decrease or increase of their serum CA 27.29 levels at week 12, relative to their baseline antigen level classification. The results were similar for both treatment arms and are shown in Table 6.

### Stratification Adjustments and Subsequent Efficacy Outcomes

In order to more fully understand the data, a patient-by-patient review was conducted following the primary analyses. One of the goals of this review was to identify whether any patients had been inadvertently mis-stratified at the time of study enrollment. Data relevant to the determination of appropriate strata assignment was compiled and reviewed in an independent and blinded fashion by the team of physicians from the collaborating sponsor companies, EMD Pharmaceuticals and Biomira. Each physician provided comments regarding the strata assignment for each patient based upon pre-determined criteria. In terms of concomitant hormone therapy use, the criteria required that patients must receive the therapy for a minimum of three months from the time of treatment arm randomization, and that the therapy used must clearly be indicated for cancer treatment, and not for symptom control. Mis-stratifications were identified in all four pre-planned subsets. The team of physicians approved the final, adjusted patient assignments, and the subsequent dataset was termed “fully adjusted”.

The numbers of patients assigned to a new stratum were as follows: 31 patients changed to the hormone group, 10 changed to the non-hormone group, 23 changed to the NPD group, and 12 changed to the NED group. Efficacy analyses were re-run on this fully adjusted dataset, and are shown in Table 7.

Following stratification adjustments, each of the efficacy analyses remained non-statistically significant. One important difference was observed however, between the results prior to and following the adjustment: the benefit observed for the THERATOPE® vaccine treated patients within the hormone subset was now seen in both the NED and NPD strata.

In an effort to understand these observations more fully, a comparison of prognostic variables between the treatment groups and within the strata was undertaken. No statistically significant differences were identified in any of the categories examined, with the exception of one variable within the NED/no hormone strata combination. In this group, CA 27.29 was significantly different, and this difference favored the KLH control patients (15.6% of KLH control patients had elevated CA 27.29 versus 51.4% of THERATOPE® vaccine treated patients; Chi sq=0.0020). When comparing all of the NED patients, regardless of concomitant hormone treatment, 43% and 21% of THERATOPE® vaccine and KLH control patients respectively, had elevated baseline serum CA 27.29.

As the number of patients who were NED at study entry was relatively small (less than 10% of all patients), subsequent strata based analyses focused primarily on examining data by hormone use (with disease status included as an explanatory variable for statistical adjustment).

A total of 180 THERATOPE® vaccine treated patients and 170 KLH control patients were included in the concomitant hormone therapy strata following the stratification adjustments. For this subset of patients who received concomitant hormone therapy, those treated with THERATOPE® vaccine survived a median 35.0 months, while KLH control patients survived a median 30.7 months (Cox p=0.1161).

FIG. 1 shows the Kaplan-Meier survival curve for these groups, utilizing the fully adjusted dataset. Although the results are not statistically significant, they are clinically encouraging.

The fully adjusted dataset was also examined to determine if a survival difference was observed between THERATOPE® vaccine and KLH control patients who did not receive concomitant hormone therapy. No significant difference was identified (18.7 and 19.6 months respectively, Cox p=0.2291).

### Survival and TDP by Antibody Response for the Fully Adjusted Hormone Subset

Survival by antibody response was also examined for the hormone subset of patients utilizing the fully adjusted dataset. The pattern and degree of benefit was similar to that found in the analysis conducted prior to the stratification adjustment and are shown in Table 8.

TDP by antibody response was also examined for the fully adjusted hormone subset. While no statistically significant differences were observed, a trend to benefit was found for THERATOPE® vaccine treated patients in terms of IgG anti-OSM response at or above the median compared to patients with responses below the median (10.6 versus 6.3 months respectively, Cox p=0.0781).

Interestingly, while the prognostic variables were well balanced in the THERATOPE® vaccine patients in terms of KLH response groups, this was not entirely the case when examining the balance of these prognostic variables in the STn or OSM response groups.

In the case of STn response, 75% of patients with lower than the median IgG response had visceral metastasis compared to 67% with responses at or higher than the median (each of the other prognostic variables were relatively balanced).

In the case of OSM response, several prognostic indicators were observed that favored patients with responses at or higher than the median, compared to those with responses below the median. These include fewer patients with visceral metastases (65% versus 82%), more patients with normal CA 27.29 at baseline (56% versus 31%), and more patients with ER+ tumors (84% versus 78%). These factors may have contributed to the ability of patients to develop higher antibody responses to OSM, as well as to the extent of survival observed. However, when these three factors were added as explanatory variables to the Cox model, the survival difference remained statistically significant (Cox p=0.0397).

KLH control patients were also observed to have an imbalance in prognostic variables between those with antibody responses at or above median and those with responses below the median. Specifically, patients with antibody responses below the median had fewer visceral metastases (71% versus 80%) and more frequent normal CA 27.29 (58% versus 41%). When these factors are added as explanatory variables to the Cox model, the survival difference remains statistically significant (Cox p=0.0357).

### Survival Updates 1 and 2

Updated survival information was analyzed on two occasions. Update #1 was six months after the endpoint for the data above, and Update #2 was six months after update #1. As the data matured and were analyzed at these additional time points, the efficacy outcomes between the overall ITT treatment groups remained essentially unchanged. These data are shown in Table 9, along with the original data provided for reference.

The survival of the hormone subset from the ITT population is shown in Table 10, along with the identical analyses for the fully adjusted hormone subset. Included in this table is the original data for reference.

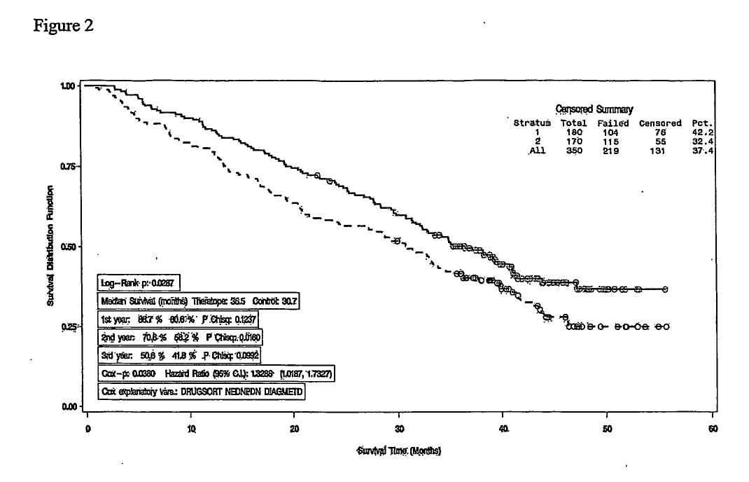

Although the statistical outcome for survival for the ITT hormone subset improves marginally over time, the analyses for the fully adjusted hormone subset of patients reached statistical significance at the most recent update (Cox p=0.0360). These data are illustrated in FIG. 2.

### Survival by Antibody Response over Time for the ITT Treatment Groups Updates 1 and 2

Survival was also assessed by antibody response over time for the overall ITT patients. The observed statistical trend for THERATOPE® vaccine treated patients with anti-OSM responses at or greater than the median, was lost over time. No significant difference in survival by antibody response to STn or KLH was observed at any time point for THERATOPE® vaccine patients.

KLH control patients with lower than median antibody responses to KLH continued to experience a survival benefit over those with a response at or higher than median, although the degree of statistical significance lessened over time. The data are shown in Table 11.

### Survival by Antibody Response over Time for the Fully Adjusted Hormone Subset: Updates 1 and 2

The analysis of survival by antibody response for the fully adjusted hormone subset of women yielded interesting observations.

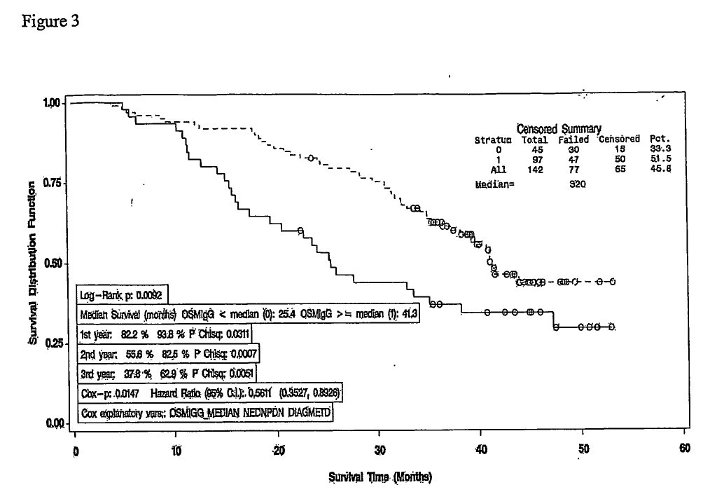

THERATOPE® vaccine treated patients with responses at or higher than the median to OSM, compared to those with less than median response to OSM, were observed to have a statistically significant survival benefit at the original analysis in June 2003. At each of the follow-up survival analyses, this benefit remained statistically significant. These data are shown in Table 12, and the outcome from the most recent survival update (#2) is illustrated in FIG. 3.

A similar outcome did not occur for KLH control patients. Although KLH control patients with lower than median antibody responses, compared to those with responses at or greater than the median, were observed to have a statistically significant survival benefit at the original analysis time point, the benefit was lost over time. The data are shown in Table 12.

### Discussion

Several observations were identified from the exploratory analyses: 1) patients who receive anti-cancer hormone therapy concomitantly with THERATOPE® vaccine vaccine have better TDP and survival outcomes than similar patients treated with KLH control vaccine, 2) no difference in TDP or survival was identified between THERATOPE® vaccine and KLH control patients who did not receive concomitant hormone therapy, 3) THERATOPE® vaccine treated patients who mount IgG anti-OSM antibody responses at or greater than the median have improved survival compared to those with less than median responses, and 4) KLH control patients with less than median IgG anti-KLH responses had improved survival compared to those with higher responses.

Not understood is the rationale behind the survival benefit observed for KLH control patients with less than median antibody responses compared to their counterparts with higher responses. Patients in the former group were observed to have several prognostic variables in their favor, which could facilitate a better survival outcome, but it remains unclear why this potentially healthier group would develop lower antibody responses to KLH than those with poorer prognostic characteristics.

The beneficial outcome associated with THERATOPE® vaccine and concomitant anti-cancer hormone therapy is not fully understood. One possible explanation for this outcome is that patients who received concomitant hormone treatment may have had a less aggressive form of breast cancer, as disease progression generally did not occur as quickly for these patients as for those who did not receive hormone therapy. This difference allowed a greater opportunity for concomitant hormone therapy patients to receive more THERATOPE® vaccine vaccinations, and therefore increase the likelihood of immune response development against their disease. While the survival benefit may be due, at least in part, to favorable imbalances in prognostic variables, it is also plausible that those same variables may also have identified patients most likely to respond favorably to immunotherapeutic approaches. Additionally, there may be some type of interaction between hormone therapy and THERATOPE® vaccine treatment.

### GLOSSARY OF TERMS

Exploratory Analysis: analysis that extends beyond the requirements stated in the trial protocol.

Intent To Treat (ITT): Includes randomized patients according to the treatment group they were randomly assigned, and the strata to which they were originally designated, prior to corrections to the stratification variables that occurred after data review.

Fully Adjusted: Corrections or adjustments are made to the stratification variables following data review.

ITT Hormone Subset: The total patient population selected for hormone use prior to corrections to the stratification variables.

Fully Adjusted Hormone Subset: The total patient population selected for hormone use following the corrections to the stratification variables.

Time To Disease Progression (TDP): Time from the date of randomization to the first reported data of disease progression if a patient has progressed or if a patient has not progressed, the date of death or if a patient is alive, the date of last contact or date lost to follow-up.

Survival Time: The difference in time from date of randomization to death, if deceased, or to date of last contact or date lost to follow-up, if alive.

Censored Observation: The last date of contact for patients still alive or lost to follow-up.

Cox Proportional Hazards Model (Cox p): A regression model for modeling survival times. The model assumes that the underlying hazard rate is a function of the independent variables and consistent over time.

Explanatory Variables: (Covariates) Sometimes called independent variables. Variables used to explain the variation in the survival time, for instance disease status, time between diagnosis and metastasis, baseline CA27.29 level, and so on.

Kaplan-Meier Survival: Estimate of the survival function for nonparametric samples, taking into account censored observations.

Log-Rank Statistic: Test for equality of the survival curves across the strata.

Kaplan-Meier Survival Median: The time at which half the subjects have reached the event of interest (normally either death or disease progression, depending on what is being analyzed), taking into account censored observations.

Probability: In the context of this analysis, the likelihood that the results seen could have occurred purely by chance.

Statistically Meaningful: When the probability is ≦0.05.

### REFERENCES

MacLean G D, Reddish M A, Koganty R R and Longenecker B M. Antibodies against mucin-associated Sialyl-Tn epitopes correlate with survival of metastatic adenocarcinoma patients undergoing active specific immunotherapy with synthetic STn vaccine. J. Immunotherapy, 1996. 19:59-68.
Chan D W, Beveridge R A, Muss H, Fritsche H A, Hortobagyi G, Theriault R, Kiang D, Kennedy B J, and Evelegh M. Use of Truquant B R radioimmunoassay for early detection of breast cancer recurrence in patients with stage II and stage III disease. J. Clin Oncol., 1997. Vol 15, 2322-2328.
Cheli C D, Morris, D L, Kish L, et al. Multicenter evaluation of the Bayer Immuno I™ CA 15-3™ assay. Clin Chem., 1998. 44:4, 765-772.
Bast R, Ravdin P, Hayes D, et al. 2000 update of recommendations for the use of tumor markers in breast and colorectal cancer: clinical practice guidelines of the American Society of Clinical Oncology. J. Clin Oncol., 2001. Vol 19, 1865-1878.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

In addition to those set forth elsewhere, the following references are hereby incorporated by reference, in their most recent editions as of the time of filing of this application: Kay, Phage Display of Peptides and Proteins: A Laboratory Manual; the John Wiley and Sons Current Protocols series, including Ausubel, Current Protocols in Molecular Biology; Coligan, Current Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manual; Manipulating the Mouse Embryo: A Laboratory Manual; Methods in Yeast Genetics: A Cold Spring Harbor Laboratory Course Manual; Drosophila Protocols; Imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons: The Experimental Basis of Neuroscience; Genome Analysis: A Laboratory Manual Series; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast Genetics: A Laboratory Course Manual; Molecular Biology of Plants: A Laboratory Course Manual.

All references cited herein, including journal articles or abstracts, published, corresponding, prior or otherwise related U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass (e.g., a disclosed class with one or more disclosed members omitted) or subrange contained therein, as well as a separate description of each individual member or value in said class or range.

The description of preferred embodiments individually shall be deemed a description of any possible combination of such preferred embodiments, except for combinations which are impossible (e.g, mutually exclusive choices for an element of the invention) or which are expressly excluded by this specification.

If an embodiment of this invention is disclosed in the prior art, the description of the invention shall be deemed to include the invention as herein disclosed with such embodiment excised.

The invention, as contemplated by applicant(s), includes but is not limited to the subject matter set forth in the appended claims, and presently unclaimed combinations thereof. It further includes such subject matter further limited, if not already such, to that which overcomes one or more of the disclosed deficiencies in the prior art. To the extent that any claims encroach on subject matter disclosed or suggested by the prior art, applicant(s) contemplate the invention(s) corresponding to such claims with the encroaching subject matter excised.

All references, including patents, patent applications, books, articles, and online sources, cited anywhere in this specification are hereby incorporated by reference, as are any references cited by said references.

TABLE 1
ITT TDP and Survival by Strata (months)

| | Median TDP | | | Median Survival | | |
|---|---|---|---|---|---|---|
| | THERATOPE ® | | Cox | THERATOPE ® | | Cox |
| Strata | vaccine | Control | p-value | vaccine | Control | p-value |
| NED/No | 2.9 (n = 40) | 2.7 (n = 38) | 0.8882 | 22.5 (n = 40) | 27.3 (n = 38) | 0.1070 |
| Hormone | | | | | | |
| NED/ | 15.8 (n = 16) | 16.5 (n = 18) | 0.9529 | 41.1 (n = 16) | Not reached | 0.8845 |
| Hormone | | | | | (n = 18) | |
| NPD/No | 2.8 (n = 315) | 2.8 (n = 306) | 0.4291 | 18.9 (n = 315) | 19.0 (n = 306) | 0.6604 |
| Hormone | | | | | | |
| NPD/ | 8.1 (n = 152) | 5.6 (n = 143) | 0.4882 | 34.3 (n = 152) | 30.1 (n = 143) | 0.2035 |
| Hormone | | | | | | |

TABLE 2
ITT TDP and Survival by Hormone Group (months)

| | Median TDP | | | Median Survival | | |
|---|---|---|---|---|---|---|
| | THERATOPE ® | | Cox | THERATOPE ® | | Cox |
| Strata | vaccine | Control | p-value | vaccine | Control | p-value |
| No Hormone | 2.8 (n = 355) | 2.8 (n = 344) | 0.5233 | 19.2 (n = 355) | 20.5 (n = 344) | 0.4084 |
| Hormone | 8.2 (n = 168) | 5.7 (n = 161) | 0.4779 | 34.9 (n = 168) | 30.7 (n = 161) | 0.2228 |

TABLE 3
ITT Overall and by Hormone Group Antibody
Responses at Week 12
Week 12 Median Titers

| | Anti- | Anti- | Anti- | Anti- | Anti- | Anti- |
|---|---|---|---|---|---|---|
| | STn | STn | OSM | OSM | KLH | KLH |
| | IgG | IgM | IgG | IgM | IgG | IgM |
| Theratope | 20480 | 10240 | 320 | 1280 | 20480 | 80 |
| Theratope H | 20480 | 10240 | 320 | 1280 | 20480 | 80 |
| Theratope | 10480 | | | | 20480 | 160 |
| NH | | | | | | |
| Control | 0 | 0 | 0 | 0 | 81920 | 1280 |
| Control H | 0 | 0 | 0 | 0 | 81920 | 1280 |
| Control NH | 0 | 0 | 0 | 0 | 81920 | 1280 |

TABLE 4
ITT Overall Median Survival by Week 12 IgG
Antibody Response (months):

| | |
|---|---|
| | KLH |
| | Control |
| THERATOPE ® vaccine N = 364 | N = 323 |

| ITT | a-STn IgG | a-OSM IgG | a-KLH-IgG | a-KLH IgG |
|---|---|---|---|---|
| =median | 31.4 | 31.2 | 31.2 | 23.9 |
| response | (n = 208) | (n = 240) | (n = 205) | (n = 204) |
| <median | 26.3 | 24 | 26.3 | 33.3 |
| response | (n = 156) | (n = 124) | (n = 159) | (n = 119) |
| Cox p- | 0.4116 | 0.0896 | 0.3183 | 0.0010 |
| value | | | | |

(“a-” means “anti-”).

TABLE 5
Survival by Antibody Response in the Hormone
Subset of Patients (months)

| | | | | KLH |
|---|---|---|---|---|
| | | | | Control |
| | | | | N = 117 |
| Hormone | THERATOPE ® vaccine N = 133 | | | a-KLH |
| subset | a-STn IgG | a-OSM IgG | a-KLH-IgG | IgG |
| =median | 41.1 | 39.6 | 35.4 | 29.3 |
| response | n = 79 | n = 93 | n = 80 | n = 70 |
| <median | 32.9 | 24.0 | 38.2 | NR |
| response | n = 54 | n = 40 | n = 53 | n = 47 |
| Cox p-value | 0.1408 | 0.0033 | 0.7579 | 0.0102 |

TABLE 6
ITT Overall CA 27.29: Change from Baseline to
Week 12

Change at
Week 12 Relative
to Baseline

| Treatment | Status at | Decrease N | Increase N |
|---|---|---|---|
| Group | Baseline | (%) | (%) |
| THERATOPE ® | Elevated | 46 (33.8) | 90 (66.2) |
| vaccine | | | |
| | Normal | 36 (26.9) | 98 (73.1) |
| KLH Control | Elevated | 41 (35.3) | 75 (64.7) |
| | Normal | 27 (23.5) | 88 (76.5) |

TABLE 7
Fully Adjusted TDP and Survival by Strata
(months)

| | Median TDP | | | Median Survival | | |
|---|---|---|---|---|---|---|
| | (months) | | | (months) | | |
| | | | Cox p | | | Cox p |
| Strata | Ther | Control | value | Ther | Control | value |
| NED/No Hormone | 2.9 | 3.0 | 0.4808 | 24.7 | 32.1 | 0.3219 |
| | (n = 36) | (n = 33) | | (n = 36) | (n = 33) | |
| NPD/No Hormone | 2.8 | 2.8 | 0.7992 | 18.2 | 18.6 | 0.2954 |
| | (n = 307) | (n = 302) | | (n = 307) | (n = 302) | |
| NED/Hormone | 19.5 | 13.7 | 0.5991 | 41.1 | 27.2 | 0.3011 |
| | (n = 16) | (n = 16) | | (n = 16) | (n = 16) | |
| NPD/Hormone | 8.2 | 5.7 | 0.3082 | 34.3 | 30.7 | 0.2085 |
| | (n = 164) | (n = 154) | | (n = 164) | (n = 154) | |

Ther = THERATOPE ® vaccine.

TABLE 8
Survival by Antibody Response in the Fully
Adjusted Hormone Subset (months)

| | THERATOPE ® vaccine | | | KLH Control |
|---|---|---|---|---|
| Hormone | N = 142 | | | N = 123 |
| sub set | a-OSM IgG | a-STn IgG | a-KLH IgG | a-KLH IgG |
| =median | 39.6 | 41.1 | 35.0 | 29.3 |
| response | n = 97 | n = 81 | n = 86 | n = 75 |
| <median | 25.4 | 35.0 | 38.2 | NR |
| response | n = 45 | n = 61 | n = 56 | n = 48 |
| Cox p-value | 0.0050 | 0.2183 | 0.9390 | 0.0072 |

TABLE 9
Median Survival for ITT Patients Over Time (months)

| | Survival | | |
|---|---|---|---|
| | THERATOPE ® | | |
| | vaccine | Control | Cox p |
| ITT | (n = 523) | (n = 505) | value |
| Original | 23.1 | 22.3 | 0.9158 |
| Update #1 | 23.1 | 22.3 | 0.8786 |
| Update #2 | 23.1 | 22.3 | 0.8732 |

TABLE 10
Median Survival for ITT and Fully Adjusted
Hormone Subsets Over Time (months)

| | Survival (months) | | |
|---|---|---|---|
| | THERATOPE ® | | |
| | vaccine | KLH Control | Cox p |
| ITT Hormone Subset | (n = 168) | (n = 161) | value |
| Original | 34.9 | 30.7 | 0.2228 |
| Update #1 | 35.4 | 31.6 | 0.2080 |
| Update #2 | 35.1 | 31.4 | 0.1386 |
| Fully Adjusted | THERATOPE ® | KLH Control | Cox p |
| Hormone Subset | vaccine | (n = 170) | value |
| | (n = 180) | | |
| Original | 35.0 | 30.7 | 0.1161 |
| Update #1 | 38.2 | 30.7 | 0.0769 |
| Update #2 | 36.5 | 30.7 | .0360 |

TABLE 11
ITT Survival by Antibody Response Over Time (months)

| THERATOPE ® vaccine (N = 364) | | | | | | | KLH Control (N = 323) | | | | | |
|---|---|---|---|---|---|---|---|---|---|---|---|---|
| | a-OSM IgG | | | a-STn IgG | | | a-KLH IgG | | | a-KLH IgG | | |
| | Median | | | Median | | | Median | | | Median | | |
| | response | | | response | | | response | | | response | | |
| Date | < | ≧ | Cox P | < | ≧ | Cox P | < | ≧ | Cox P | < | ≧ | Cox P |
| Orig | 24.0 | 31.2 | 0.0896 | 26.3 | 31.4 | 0.4116 | 26.3 | 31.2 | 0.3183 | 33.3 | 23.9 | 0.0010 |
| Upd 1 | 24.0 | 31.4 | 0.2246 | 26.4 | 31.4 | 0.7595 | 25.9 | 31.2 | 0.4312 | 32.7 | 23.9 | 0.0101 |
| Upd 2 | 24.0 | 31.2 | 0.4179 | 26.4 | 31.4 | 0.8269 | 25.9 | 31.4 | 0.5919 | 32.7 | 23.9 | 0.0455 |

TABLE 12
Survival by Antibody Response for the Fully Adjusted Hormone Subset Over Time
(months)

| THERATOPE ® vaccine (N = 142) | | | | | | | KLH Control (N = 123) | | | | | |
|---|---|---|---|---|---|---|---|---|---|---|---|---|
| | a-OSM IgG | | | a-STn IgG | | | a-KLH IgG | | | a-KLH IgG | | |
| | Median | | | Median | | | Median | | | Median | | |
| | response | | | response | | | response | | | response | | |
| | < n = | = n = | | < n = | = n = | | < n = | = n = | | < n = | = n = | |
| Date | 45 | 97 | Cox P | 61 | 81 | Cox P | 56 | 86 | Cox P | 48 | 75 | Cox P |
| Orig | 25.4 | 39.6 | 0.0050 | 35.0 | 41.1 | 0.2183 | 38.2 | 35.0 | 0.9390 | NR | 29.3 | 0.0072 |
| Upd 1 | 25.4 | 41.1 | 0.0125 | 35.0 | 41.1 | 0.3610 | 39.6 | 41.1 | 0.9118 | 43.0 | 31.6 | 0.0657 |
| Upd 2 | 25.4 | 41.3 | 0.0147 | 35.1 | 41.1 | 0.3745 | 38.2 | 40.8 | 0.9346 | 39.5 | 30.8 | 0.3387 |

NR = Not reached.

1. A method of treating breast cancer which comprises administering, to a subject suffering from breast cancer,
a first amount of anti-estrogenic steroid agent, effective to reduce the level or activity of at least one estrogenic steroid in the subject, and
a second amount of an immunological agent, effective to contribute to the development of a protective immune response to said breast cancer,
where said first and second amounts are, at least in combination, therapeutically effective against at least some breast cancers.

2. The method of claim 1 where said agents are administered concurrently.

3. The method of claim 1 where said immunological agent comprises at least one immunogen, said immunogen comprising at least one breast cancer-associated epitope.

4. The method of claim 3 where at least one epitope is a MUC1 epitope.

5. The method of claim 3 where at least one epitope is a carbohydrate epitope.

6. The method of claim 3 in which said immunogen comprises STn.

7. The method of claim 6 in which said immunogen comprising STn is an STn-KLH conjugate.

8. The method of claim 7 in which the conjugate is an aggregated conjugate.

9. The method of claim 7 in which the conjugate has a NANA content of about 7%.

10. The method of claim 1 in which the anti-estrogenic steroid agent comprises at least one antiestrogen.

11. The method of claim 10 in which at least one antiestrogen is a steroidal antiestrogen.

12. The method of claim 1 in which at least one anti-estrogenic steroid agent is fulvestrant.

13. The method of claim 10 in which at least one antiestrogen is a nonsteroidal antiestrogen.

14. The method of claim 13 in which at least one nonsteroidal antiestrogen is selected from the group consisting of toremifene, tamoxifen, droloxifene and trioxifene.

15. The method of claim 1 in which the anti-estrogenic steroid agent comprises at least one aromatase inhibitor.

16. The method of claim 15 in which at least one aromatase inhibitor is selected from the group consisting of aminoglutethimide, anastrozole, vorozole, letrozole, liarozole, megastrole, exemestane and formestane.

17. The method of claim 1, further comprising administration of at least one progestin which protects against breast cancer.

18. The method of claim 17 in which at least one progestin is progesterone.

19. The method of claim 1, further comprising administration of at least one anti-progestin which protects against breast cancer.

20. The method of claim 1 in which the anti-estrogenic steroid agent comprises geoselin acetate or megestrol acetate.

21. The method of claim 1 in which the combination of the anti-estrogenic steroid agent and the immunological agent is synergistically effective against breast cancer.

22. The method of claim 1, further comprising administration of a therapeutically effective amount of at least one chemotherapeutic agent other than an anti-estrogenic steroid agent.

23. The method of claim 22 in which at least one chemotherapeutic agent is an anthracycline.

24. The method of claim 23 in which at least one anthracycline is selected from the group consisting of doxorubicin, daunorubicin, epirubicin, and idarubicin.

25. The method of claim 22 in which at least one chemotherapeutic agent is a taxane.

26. The method of claim 25 in which at least one taxane is paclitaxel or docetaxel.

27. The method of claim 1 in which the anti-estrogenic steroid agent comprises at least one compound which antagonizes at least one estrogen receptor by competitively inhibiting the binding of an estrogen to that receptor without itself activating that receptor.

28. The method of claim 27 in which said receptor antagonist is not an agonist for any estrogen receptor.

29. The method of claim 27 in which said receptor inhibitor is also an agonist of at least one other estrogen receptor, and consequently is a SERM.

30. The method of claim 29 in which said SERM is selected from the group consisting of tamoxifen, toremifene, droloxifen, clomifene, arzoxifene, raloxifene, LY 117018 and SERM EM-652.

31. The method of claim 1 in which the breast cancer is a metastatic breast cancer.

32. A therapeutic composition comprising (a) at least one anti-estrogenic steroid agent, and (b) at least one immunogenic agent, which, when administered according to a suitable therapeutic schedule, is therapeutically effective against breast cancer.

33. A kit comprising a first container comprising at least one dose of at least one anti-estrogenic steroid agent, and a second container comprising at least one dose of at least one immunogenic agent, where said agents are, at least in combination, therapeutically effective against breast cancer.

34-37. (canceled)

38. The composition of claim 32, wherein said immunogenic agent comprises at least one immunogen, said immunogen comprising at least one breast cancer-associated epitope, and said anti-estrogenic steroid agent comprises at least one antiestrogen.

39. The kit of claim 33, wherein said immunogenic agent comprises at least one immunogen, said immunogen comprising at least one breast cancer-associated epitope, and said anti-estrogenic steroid agent comprises at least one antiestrogen.
